(19) United States
(12) Patent Application Publication
(10) Pub. No.: US 2014/0093525 A1
(43) Pub. Date: Apr. 3, 2014

(54) COMPOSITIONS AND METHODS OF TREATING OPIOID ADDICTION

(71) Applicants: Minneapolis Medical Research Foundation, Minneapolis, MN (US); REGENTS OF THE UNIVERSITY OF MINNESOTA, St. Paul, MN (US)

(72) Inventors: Paul Richard Pentel, Minneapolis, MN (US); Philip S. Portoghese, St. Paul, MN (US); Marco Pravetoni, Minneapolis, MN (US); Morgan C.P. Le Naour, St. Paul, MN (US)

(73) Assignees: Minneapolis Medical Research Foundation, Minneapolis, MN (US); REGENTS OF THE UNIVERSITY OF MINNESOTA, St. Paul, MN (US)

(21) Appl. No.: 14/029,568

(22) Filed: Sep. 17, 2013

Related U.S. Application Data
(60) Provisional application No. 61/702,126, filed on Sep. 17, 2012.

(51) Int. Cl. A61K 39/385

(52) U.S. Cl. CPC A61K 39/385
USPC 424/185.1; 530/330

(57) ABSTRACT

The present invention relates to conjugates and vaccine compositions for treatment of opioid addiction, and methods of use of these conjugates and compositions.

### CROSS-REFERENCE TO RELATED APPLICATIONS

This patent application claims the benefit of priority of U.S. provisional application Ser. No. 61/702,126, filed Sep. 17, 2012, which application is herein incorporated by reference.

### FEDERAL GRANT SUPPORT

This invention was made with government support under Grant No. DA026300 awarded by the National Institutes of Health. The government has certain rights in the invention.

### BACKGROUND OF THE INVENTION

Opioids were the first target for addiction vaccines and provided proof-of principle that this approach can attenuate addictive behavior. Heroin/morphine vaccines have been developed in the past and were shown to be immunogenic and capable of binding and retaining morphine in serum. Limitations of the existing opioid vaccine work are that these vaccines have undergone only preliminary investigation of their immunogenicity, effects on opioid pharmacokinetics, effects on opioid-induced behaviors, and the dose-response relationships important for predicting clinical efficacy (opioid dose size, acute v. chronic dosing). Moreover, these vaccines were directed at heroin and its metabolites and did not address other commonly abused opioids. Many other opioids besides heroin are also abused, and individuals may switch between opioids. An opioid vaccine that can reduce or attenuate the effects of multiple different opioids could be useful by providing additional activity against a wider range of opioids compared to vaccines that target only heroin and its metabolites.

### SUMMARY OF THE INVENTION

In certain embodiments, the present invention provides a multivalent composition comprising a mixture of at least two different antigenic opioid conjugates, comprising:
Hapten¹-X¹-Z¹,
Hapten²-X²-Z², and optionally
Hapten³-X³-Z³,
wherein Hapten¹ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, or another opioid;
wherein Hapten² is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, or another opioid but is not the same compound as Hapten¹ or Hapten³,
wherein Hapten³ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, or another opioid, but is not the same compound as Hapten¹ or Hapten²,
wherein X¹, X² and X³ are linking groups, and
wherein Z¹, Z² and Z³ are antigenic carrier molecules.

In certain embodiments additional conjugates are included in the same vaccine formulation to increase the diversity of opioids targeted by the multivalent vaccine.

In certain embodiments, X¹ and/or X² and/or X³ is a peptide, such as a peptide that is 2-8 amino acids in length, such as (Gly)₄. In certain embodiments, Z¹ and/or Z² and/or Z³ is a protein or peptide. In certain embodiments, Z¹ and/or Z² and/or Z³ is keyhole limpet hemocyanin (KLH) or cholera toxin B (rCTB) or recombinant diphtheria toxin CRM197 or tetanus toxoid (TT). In certain embodiments, Hapten¹ is a derivative of oxycodone. In certain embodiments, the multivalent composition elicits an immune response against the haptens. In certain embodiments, the multivalent composition induces antibody production in a mammal, wherein the antibodies have sufficient affinity for an opioid to alter the opioid's distribution to brain or behavioral effects. In certain embodiments, the antibodies have a Kd of <50 nM for the haptens. In certain embodiments, the multivalent composition elicits antibodies that have a lower affinity for methadone, buprenorphine, naloxone, naltrexone or endogenous opioids than for their targeted opioids. In certain embodiments, the multivalent composition elicits antibodies that have a Kd of >50 mM for methadone, buprenorphine, naloxone, naltrexone or endogenous opioids. In certain embodiments, the vaccine composition includes an adjuvant such as alum or monophosphoryl lipid a (mpla) or other suitable adjuvant or combinations of adjuvants. In certain embodiments, the multivalent composition comprises two or more (e.g., three, four, five or six) different antigenic opioid conjugates.

In certain embodiments, the present invention provides antigenic opioid conjugate comprising:
Hapten-X-Z
wherein the hapten is a derivative of heroin, 6-acetylmorphine, morphine-6-glucuronide, hydromorphone, codeine, oxymorphone, hydrocodone, fentanyl, sufentanil, meperidine, or alfentanil,
wherein X is a linking group, and
wherein Z is an antigenic carrier molecule.

In certain embodiments, X is a peptide, such as a peptide that is 2-8 amino acids in length. In certain embodiments, X is (Gly)₄. In certain embodiments, Z is a protein or peptide. In certain embodiments, Z is KLH or rCTB or CRM197. In certain embodiments, the Hapten is oxycodone or morphine. In certain embodiments, the Hapten is a derivative of oxycodone. In certain embodiments, the conjugate elicits an immune response against the hapten. In certain embodiments, the conjugate induces antibody production in a mammal, wherein the antibodies have sufficient affinity for opioid to alter the opioid's distribution or its behavioral effects in the mammal. In certain embodiments, the conjugate induces antibody production in a mammal, wherein the antibodies have a dissociation constant of Kd of <50 nM for each of the haptens. In certain embodiments, the conjugate elicits antibodies that have a lower affinity for methadone than for the targeted opioid.

In certain embodiments, the present invention provides antigenic opioid conjugate comprising:
Oxycodone-X-Z
wherein X is a linking group, and
wherein Z is an antigenic carrier molecule.

In certain embodiments, the present invention provides a vaccine formulation comprising an effective amount of the antigenic composition or the antigenic opioid conjugate described above. In certain embodiments the present inventions provides a vaccine formulation comprising a mixture of 2 or more conjugates which effects of each to be obtained concurrently.

In certain embodiments, the present invention provides a method of treating opioid addiction comprising administering to a mammal in need thereof the composition or the antigenic opioid conjugate described above, wherein the serum level of generated antibodies is sufficient to alter the opioid's distribution or its behavioral effects in the mammal. In certain embodiments, the present invention provides a method of treating opioid addiction comprising administering to a mammal in need thereof the composition or the antigenic opioid conjugate described above, wherein serum level of generated antibodies is >100 μg/ml. In certain embodiments, the serum level of generated antibodies is such that the level of opioid reaching the brain is diminished by at least 10%. In certain embodiments, a serum level of generated antibodies of >100 μg/ml is maintained in the mammal for at least one month. In certain embodiments, the method further comprises administering an opioid receptor antagonist such as naltrexone (NLTX). In certain embodiments, the method further comprises administering a pharmaceutical, wherein the pharmaceutical is methadone or buprenorphine.

In certain embodiments, the present invention provides a purified antibody that binds to an opioid conjugate or to the target opioid itself. In certain embodiments, the antibody is a human antibody or a humanized antibody. In certain embodiments, the antibody is a single-chain Fv or an scFv fragment.

In certain embodiments, the present invention provides a method of treating an opioid overdose comprising administering the purified antibody described above.

In certain embodiments, the present invention provides a method of detecting the presence of an opioid in a sample comprising contacting the sample with the purified antibody described above.

### BRIEF DESCRIPTION OF DRAWINGS

FIG. 1. Effects of two vaccine doses on OXY distribution. Rats immunized with either 25 or 100 μg of conjugate (five per group) received 0.5 mg/kg i.v. OXY, and samples were collected 5 min later. A and B, data (mean±S.D.) represent the total OXY concentration in serum or brain for animals immunized with OXY(Gly)₄-BSA (A) or OXY(Gly)₄-KLH (B). Numbers over the data bars are the percentage of change compared with control. Vaccination with OXY(Gly)₄-KLH increased retention of OXY in serum and both immunogens decreased OXY distribution to brain, *, p<0.05 compared with controls. C, there was a significant correlation between the log serum antibody titer and the brain OXY concentration. , values for all rats receiving either conjugate vaccine shown in A and B; ∘, mean±S.D. for control rats from those experiments.

FIG. 2. Effect of vaccination on several OXY doses. Rats were immunized with 100 μg of OXY(Gly)₄-BSA and treated as in FIG. 1 but received different OXY doses intravenously. Effects on OXY distribution were greatest at the lower OXY doses as indicated by the percentage of control concentrations. Data are represented as mean±S.D. *, p<0.05; **, p<0.01 compared with control. #, OXY concentrations too low to quantitate.

FIG. 3. Effect of vaccination on OXY-induced analgesia. Rats were immunized with 100 μg of OXY(Gly)₄-KLH or KLH control and tested for blockage of OXY-induced analgesia on a hot-plate thermal nociception test. A, dose-response relationship for OXY effect on MPE % at 30 min after injection. B, rats immunized with OXY(Gly)₄-KLH or KLH control exhibited similar baseline latencies. C, effects of 2.25 mg/kg OXY administered subcutaneously were blunted in rats vaccinated with OXY(Gly)₄-KLH compared with controls immunized with unconjugated KLH. Data are expressed as mean * S.E.M. *, p<0.01 compared with control.

FIG. 4. Morphine, oxycodone, their immunogens and linker structures.

FIG. 5. (Upper panel) Relationship of log serum morphine-specific and oxycodone specific antibody titers in the bivalent vaccine group showing a significant positive correlation. (Lower panel) Serum 6-MAM and oxycodone concentrations in the bivalent group were also correlated.

FIG. 6. Vaccine effects on (upper panel) 6-MAM retention in serum, (middle panel) morphine retention in serum, and (lower panel) 6-MAM distribution to brain. Note 10-fold different vertical axis scales for 6-MAM and morphine serum concentrations. Numbers in parentheses are the % decrease compared to the KLH control group brain concentrations. Morphine distribution to brain is not shown because concentrations were below the assay detection limit. Data are the mean±SD. * p<0.05 compared to KLH control, # p<0.05 bivalent compared to monovalent vaccine.

FIG. 7. Vaccine effects on oxycodone concentrations in serum (upper panel) and brain (lower panel). Numbers in parentheses are the % decrease compared to the KLH control group. Data are the mean±SD, * p<0.05 compared to KLH control.

FIG. 8. Relationships between log serum antibody titers and serum or brain 6-MAM (A) or oxycodone (B) concentrations. Brain concentrations of drug for the KLH control group are also shown (white open circle). Separate regression lines are shown for monovalent and bivalent vaccines in each panel, and their slopes did not differ significantly between groups.

FIG. 9. Oxycodone, hydrocodone and haptens (derivatized opioids).

FIG. 10. Effect of immunization on oxycodone distribution after intravenous administration in rats.

Vaccine effects on (upper panel) serum and (lower panel) brain oxycodone, after administration of 0.1 mg/kg oxycodone i.v. Numbers in parentheses are the % decrease compared to the KLH control group. Data are the mean±SD. * p<0.05 compared to KLH control, # p<0.05 compared to other vaccines.

FIG. 11. Effect of immunization on oxycodone or hydrocodone distribution and analgesia after subcutaneous drug administration in rats. Vaccine effects on (panel A) serum concentrations, (panel B) brain concentrations and (panel C) analgesia, after administration of 2.25 mg/kg oxycodone s.c or 6.75 mg/kg hydrocodone s.c. Numbers in parentheses are the % decrease compared to the KLH control group. Distribution data are the mean±SD, while behavioral data are mean±SEM. * p<0.05 compared to KLH control, # p<0.05 compared to other vaccines vaccine.

FIG. 12. Effect of immunization on oxycodone analgesia in mice effects on oxycodone analgesia, after administration of 2.25 mg/kg oxycodone s.c. in BALB/c mice immunized with alum adjuvant s.c. Numbers in parentheses are the % decrease compared to the KLH control group MPE %. Data are mean±SEM. * p<0.05 compared to KLH control.

FIG. 13. Evaluation of 6OXY(S)-mKLH (8) conjugate conditions Vaccine effects on oxycodone analgesia, after administration of 2.25 mg/kg oxycodone s.c. in BALB/c mice immunized with alum adjuvant s.c. Behavioral evaluation of sulfhydryl-maleimide optimal coupling conditions, all mice are vaccinated with 6OXY(S)-mKLH (8) using in-house (left data set) or commercially activated mKLH (right data set). On the X axis, numbers are the molar ratio between hapten 5 and protein in the coupling reactions. Numbers in parentheses are the % decrease compared to the mKLH control group MPE %.

FIG. 14. Opioid compounds and derivatives.

FIG. 15. Fentanyl analgesia was measured on a hot plate at 30 minutes after injection, and the percent maximum possible effect (MPE) was calculated as (post-drug latency−baseline latency)/(maximum latency−pre-drug baseline)×100.

FIG. 16. The fixed ratio (FR) is the number of active lever presses to deliver an intravenous infusion of 0.03 mg/kg oxycodone. All rats were tested in daily 120-minute sessions. Data are expressed as mean±standard deviation. Statistical symbols: *p<0.05 compared to KLH control.

FIG. 17. Reagents. a. HBTU, DIPEA, DMF, r.t, 16 h, 93%; b. H₂, Pd(OH)₂ (10%), MeOH, r.t, 50 psi, 16 h, quantitative; c. i) HBTU, DIPEA, DCM, r.t, 16 h, 89%; ii) TFA/DCM (2/8; v/v), r.t, 87%.

FIG. 18. This figure displays a synthetic pathway that was experimented and optimized to provide the targeted haptens.

FIG. 19. This figure displays a synthetic pathway that was experimented and optimized to provide the targeted haptens.

### DETAILED DESCRIPTION OF THE INVENTION

Haptens

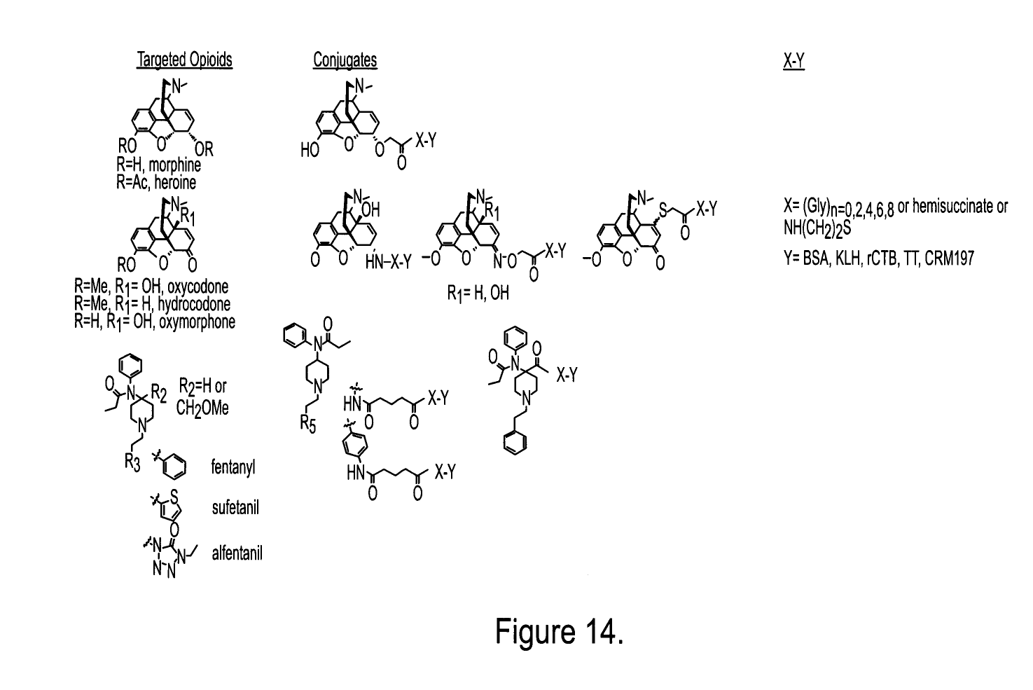

Hapten refers to an opioid with a functional group added to serve as a site for linker attachment (i.e., an opioid derivative). In certain embodiments, the hapten is a derivative of heroin, 6-acetylmorphine, morphine, morphine-6-glucuronide, hydromorphone, codeine, oxycodone, oxymorphone, hydrocodone, naloxone, naltrexone, buprenorphine, fentanyl, sufentanil, alfentanil (or another known opioid), or their active metabolites. See FIG. 14. Haptens are conjugated using linkers of various lengths to one of several carrier proteins and formulated with one or more of several adjuvants to identify the most immunogenic conjugates.

Immunogen refers to the hapten conjugated through a linker to a carrier molecule (e.g., protein or peptide), necessary because small molecules such as opioids are not immunogenic by themselves. In some embodiments immunogens are identified which elicit antibodies that do not appreciably cross react with methadone or buprenorphine, the opioid antagonists naloxone and naltrexone, or endogenous opioids. As used herein “vaccine” or “conjugate vaccine” refers to the final formulation that is administered to the subject, often consisting of an immunogen mixed with AN adjuvant. Multivalent vaccine refers to a mixture of two or more immunogens formulated with or without an adjuvant. In general, where recognition of differences in opioid structure is not desired, linkers will be attached to drug at sites where the intended target opioid structures differ. The linker then masks these sites with regard to immune recognition and the resulting immunogen elicits antibodies which bind (cross react with) each of the target opioids. Similarly, if recognition of differences is desired, the linker is placed distant from the site at which the compounds differ.

Linkers

Linkers used to conjugate or tether the haptens to carriers. Linker length can affect immunogenicity, but optimal length is difficult to predict. In certain embodiments, if the linker is a peptide, the length of the linker is between 1 and 10 amino acids. In certain embodiments, the linker length is between 5 and 20 atoms. In certain embodiments, the haptens can be tethered to carriers by bioconjugation to form a covalent bonds, such as by a peptide coupling mode of conjugation or by thiol-based maleimide conjugation.

Carriers

In certain embodiments, the hapten is conjugated or linked to a carrier molecule that is a peptide or protein. Carrier proteins commonly chosen are those known to be immunogenic when used in conjugate vaccines, and which are suitable for clinical use.

In some embodiments the protein haptenation ratio (number of hapten molecules per protein molecule) is measured with mass spectrometry and a higher haptenation ratio, for example >10 for a carrier protein of 50 Kd molecular weight, enhances immunogenicity.

Useful immunogenic proteins include keyhole limpet hemocyanin (KLH), human gamma globulin, chicken immunoglobulin G, bovine gamma globulin, tetanus toxin or toxoid, diphtheria toxin or toxoid, mutant of diphtheria toxin CRM 197, pseudomonas exotoxin A, cholera toxin or toxoid, Group A streptococcal toxins, pneumolysin of Streptococcus pneumoniae, filamentous haemagglutinin (FHA), FHA fragments of Bordetella pertussis; pili or pilins of Neisseria gonorrhoeae, pili or pilins of Neisseria meningitidis; outer membrane proteins of Neisseria meningitidis, outer membrane proteins of Neisseria gonorrhoeae; C5A peptidase of Streptococcus or a surface protein of Moraxella catarrhalis.

Adjuvants

Adjuvants can be added to the composition in certain embodiments. For example, alum or monophosphoryl lipid A (MPLA) can be used in certain embodiments. Suitable adjuvants include but are not limited to surfactants, e.g., hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, alum, and mixtures thereof. Other potential adjuvants include the B peptide subunits of E. coli heat labile toxin or of the cholera toxin, or CpG oligonucleotides.

Formulations and Modes of Administration

The present invention provides a vaccine for use to treat opioid addiction. In one embodiment of this invention, the vaccine composition can be delivered to a mammal in a pharmacologically acceptable vehicle.

To immunize a subject, the vaccine composition, is administered parenterally, usually by intramuscular or subcutaneous injection in an appropriate vehicle. Other modes of administration, however, such as oral, intranasal or intradermal delivery, are also possible.

Vaccine formulations will contain an effective amount of the active ingredient in a vehicle, the effective amount being readily determined by one skilled in the art. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the vaccine composition in one or more doses. Multiple doses may be administered as is required to achieve and maintain a state of immunity to the opioid.

To prepare a vaccine, vaccine composition is adjusted to an appropriate concentration, optionally combined with a suitable vaccine adjuvant, and packaged for use. In certain embodiments, the immunogenic product may be incorporated in or onto liposomes or other nanoparticle structures for use in a vaccine formulation.

The invention will now be illustrated by the following non-limiting Examples.

### Example 1

### An Oxycodone Conjugate Vaccine Elicits Drug-Specific Antibodies that Reduce Oxycodone Distribution to Brain and Hot-Plate Analgesia

There are an estimated 15 million users of illicit opioids worldwide (http://www.unodc.org/documents/wdr/WDR_{—}2010/World_Drug_Report_{—}2010_lo-res.pdf) and 1.2 million heroin users in the United States (http://oas.samhsa.gov/NSDUH/2k10NSDUH/2k10Results.htm). Until recently heroin use predominated in the United States, but over the past 10 years the abuse of prescription opioids has increased dramatically and is now more common than heroin abuse. The rise in prescription opioid abuse has been accompanied by a substantial increase in the incidence of emergency-department visits and fatal opioid overdoses. Oxycodone (OXY) is the most commonly abused prescription opioid (Compton and Volkow, 2006; Lopez et al., 2009). Treatment options have been developed for heroin addiction, but fewer options have been studied for abuse of OXY or other prescription opioids. Agonist therapies for heroin addiction such as methadone and buprenorphine can be very effective, but their own abuse potential and risk of side effects obligate careful and frequent monitoring, and their therapeutic use is legally restricted to those regularly using substantial quantities of opioid over a sustained period of time (Fareed et al., 2011). Many prescription opioid abusers do not fit this profile because their opioid use is oral rather than intravenous and may be sporadic, yet they still run the risk of overdose, social disruption, and transition to intravenous drug use and addiction. Additional treatment options for prescription opioid abuse are needed (Stotts et al., 2009; Dodrill et al., 2011; Maxwell, 2011).

Vaccines are being studied as a potential adjuncts to drug abuse or addiction treatment. They are of interest because they target the drug rather than the brain and therefore lack central nervous system side effects. Addictive drugs are too small to stimulate an immune response but can be rendered immunogenic by conjugation to a foreign carrier protein through a linker arm (Chi, 2011). Such conjugate vaccines stimulate the production of drug-specific antibodies that can bind their target drug in serum and extracellular fluid and reduce or slow its distribution to brain. Efficacy in blocking a wide range of addiction-like behaviors has been shown in animals for vaccines directed against nicotine, cocaine, methamphetamine, and heroin (Chi, 2011). Nicotine and cocaine conjugate vaccines have entered clinical trials with some early evidence of efficacy and no important side effects (Martell et al., 2009; Hatsukami et al., 2011). A number of morphine vaccines have been developed that produce antibodies that cross-react with heroin and its active metabolites and block or attenuate the behavioral effects of heroin or morphine in rodents. A desirable feature for such vaccines is that they not bind or block the actions of certain off-target opioids such as methadone or buprenorphine so that these can still be used therapeutically for treating opioid addiction or for analgesia (Wainer et al., 1973; Bonese et al., 1974; Anton and Leff, 2006; Anton et al., 2009; Stowe et al., 2011). Although the immunological and behavioral effects of heroin/morphine vaccines have been studied in animals, their effects on opioid pharmacokinetics, which mediate their behavioral actions, have not been reported.

The goal of the current study was to synthesize and evaluate the immunologic and pharmacokinetic effects of candidate OXY conjugate vaccines in rats. Several linkers and carrier proteins were used to assess their immunogenicity and the influence of the degree of protein haptenation on vaccine efficacy. Effects of the lead candidate OXY(Gly)₄-keyhole limpet hemocyanin (KLH) vaccine on OXY protein binding in serum, OXY distribution to brain, and OXY-induced analgesia were evaluated to provide mechanistic information and anticipate whether additional study of this vaccine is warranted. The effects of OXY(Gly)₄-KLH on OXY induced hot-plate analgesia were studied to measure the ability of this vaccine to block a centrally mediated opioid effect. The large observed effects of OXY(Gly)₄-KLH on OXY pharmacokinetics, and its ability to block OXY analgesia, support its further development as a potential adjunct to addiction treatment.

Materials and Methods

Overview of Hapten Synthesis.

The 6-position of OXY was selected for linker attachment because it has been used previously to generate effective antibodies to heroin and its active metabolites (Bonese et al., 1974; Anton and Leff, 2006). Two linkers were chosen for evaluation: hemisuccinate, because it has been used successfully with heroin conjugate vaccines, and (Gly)₄, because preliminary studies using oligoglycine as a convenient means of studying homologous linkers of different lengths suggested that (Gly)₄ was the most promising of this series (data not shown).

Compound 1 was obtained as reported previously (Lamont et al., 2003) by the condensation of OXY with O-carboxymethoxylamine hemihydrochloride (2-(aminooxy)acetic acid) in refluxing methanol using pyridine as a base (see Scheme 1). This intermediate was coupled to tetraglycine tertbutyl ester (Gly₄tBu) (Bieniarz et al., 1988), using a N,N′-dicyclohexylcarbodiimide/hydroxybenzotriazole procedure followed by acid hydrolysis to afford OXY(Gly)4 in moderate yield (52% overall). Compound 2 was prepared stereoselectively by using a catalytic reduction of the imine formed from the oxycodone and benzylamine, followed by debenzylation (Sayre and Portoghese 1980). Condensation of the intermediate 2 in a manner similar to that described previously for morphine 3-succinyl (Wainer et al., 1972) with succinic anhydride in refluxing pyridine gave OXY(HS) with good yield (82%). Compounds were characterized by 1H NMR and mass spectrometry after purification. Purity of target haptens was determined by elemental analysis.

Reagents.

All commercial reagents and anhydrous solvents were used without further purification or distillation, unless otherwise stated. Analytical thin-layer chromatography was performed on plates coated with EM Science (Gibbstown, N.J.) silica gel 60 F254 (0.25 mm). Compounds were visualized by UV light and/or stained with potassium permanganate solution followed by heating. Flash column chromatography was performed on EM Science silica gel 60 (230-400 mesh). NMR (1H) spectra were recorded on a Bruker Avance 400 MHz spectrometer (Bruker Daltonics, Billerica, Mass.) and calibrated by using an internal reference. Chemical shifts are expressed in ppm, and coupling constants (J) are in hertz (Hz). Peak multiplicities are abbreviated as: broad, br; singlet, s; doublet, d; triplet, t; and multiplet, m. ESI mode mass spectra were recorded on a BrukerBioTOF II mass spectrometer. Elemental analyses were performed by M-H-W Laboratories (Phoenix, Ariz.).

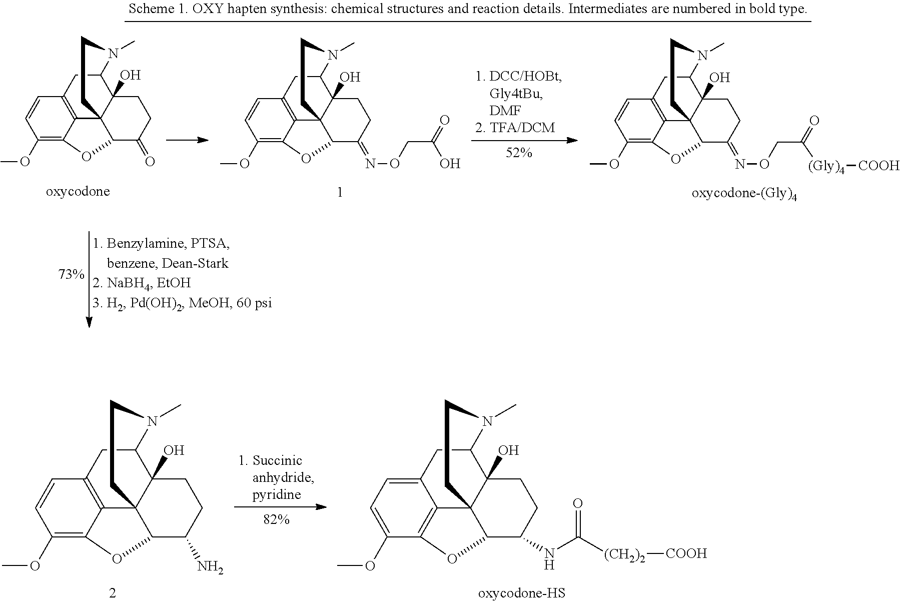

OXY(Gly)₄.

N,N′-dicyclohexylcarbodiimide (1.3 equivalent), carboxylic acid 1 (0.15-0.4 mmol scale, 1 equivalent), and hydroxybenzotriazole (1.2 equivalent) were dissolved in 5 ml of anhydrous dimethylformamide. The solution was cooled to 0° C. and placed under a nitrogen atmosphere. After 15 min at 0° C., tetraglycine (0.15-0.4 mmol, 1 equivalent) was added. The solution was sealed under a nitrogen atmosphere and allowed to warm and stir at room temperature overnight. The reaction mixture was filtered to remove dicyclohexylurea into water (10″ initial volume of dimethylformamide) and extracted with ethyl acetate. The combined organic layers were dried on magnesium sulfate, filtered, and concentrated under reduced pressure. The residue was purified by silicon dioxide (SiO₂) chromatography (DCM/MeOH/NH₄OH: 94:5:1) to afford the desired compound as a pale yellow solid (63% yield). ¹H NMR (CD₃OD) δ: 1.39 (m, 1H); 1.49 (s, 9H); 1.62-1.88 (m, 2H); 2.42-2.81 (m, 7H with s, 3H, NCH₃ at 2.68); 3.03-3.19 (m, 2H); 3.37-3.42 (m, 8H); 3.53 (d, 1H, J $9.4 Hz); 3.61 (m, 1H); 3.84 (s, 3H); 4.67 (s, 2H); 5.07 (s, 1H); 6.83 (d, 1H, J_{H1-H2} $8.1 Hz); 6.89 (d, 1H, J_{H2-H1} 8.1 Hz); ESI-TOF MS m/z: 673.789 (MH⁺).

Trifluoroacetic acid (TFA, 20% volume) was added to a solution of this ester (0.15-0.4 mmol scale) in DCM (5 ml). The resultant solution was stirred at room temperature. Upon complete disappearance of starting material, the solvent was removed under vacuum. The crude reaction mixture was subjected to azeotropic drying by using toluene. The residue was purified on a reverse-phase high-performance liquid chromatography column (Haisil 100-C18, 5 μm, 10 mm×250 mm), which was eluted with acetonitrile/H₂O/TFA (70:30:0.1) at a flow rate of 2 ml/min) to provide OXY(Gly)₄ as a white solid (82% yield). The hapten was converted to the TFA salt to facilitate purification and recovery, and TFA was then removed by ion exchange before conjugation. ¹H NMR (CD₃OD) δ: 1.42 (m, 1H); 1.69-1.91 (m, 2H); 2.48-2.82 (m, 7H with s, 3H, NCH₃ at 2.67); 3.05-3.23 (m, 2H); 3.34-3.38 (m, 8H); 3.48 (d, 1H, J $9.4 Hz); 3.61 (m, 1H); 3.85 (s, 3H); 4.63 (s, 2H); 5.04 (s, 1H); 6.81 (d, 1H, J_{H1-H2}=8.1 Hz); 6.88 (d, 1H, J_{H2-H1}=8.1 Hz); ESI-TOF MS m/z: 617.233 (MH⁺); analytical calculation for C₂₈H₃₆N₆O₁₀: C, 54.54; H, 5.88; N, 13.63. Found: C, 54.25; H, 6.03; N, 12.72.

6α-Amino-14-Hydroxydesocodeine (2).

A benzene solution containing oxycodone base (2 g, 6.3 mmol), benzylamine (770 mg, 7.15 mmol), and a catalytic amount of p-toluenesulfonic acid (5% molar) was refluxed overnight, using a Dean-Stark Trap (Sigma-Aldrich, St. Louis, Mo.). The mixture was concentrated, and a solution of NaBH₄ (43 mg, 1.1 mmol) in absolute ethanol was added (Sayre and Portoghese, 1980). After being stirred under N₂ for 8 h, the resulting solution was diluted with H₂O and concentrated to remove most of the ethanol. Additional diluted NH₄OH was added, and the aqueous layer was extracted (3×DCM). Organic layers were then dried on magnesium sulfate and concentrated under reduced pressure. This residue was purified by flash chromatography using a DCM/MeOH/NH₄OH mixture (96:3:1) to give the benzylated intermediate as a white solid (78%). ¹H NMR (CDCl₃): 1.31-1.40 (m, 2H); 1.52-1.58 (m, 2H); 2.10-2.13 (m, 2H); 2.26-2.29 (m, 2H); 2.49 (s, 3H, NCH₃); 2.54 (m, 1H); 2.91-3.02 (m, 2H); 3.30 (s, 3H, OCH3); 3.81 (s, 2H); 4.60 (d, 1H, J_{H5-H6}=3.7 Hz); 4.73 (m, 1H); 6.42 (d, 1H, J_{H1-H2}=8.1 Hz); 6.54 (d, 1H, J_{H2-H1}=8.1 Hz); 7.19-7.36 (m, 5H); ESI-TOF MS m/z: 407.538 (MH⁺).

Catalytic hydrogenolysis of the protected intermediate (1 g, 2.46 mmol) was conducted at 60 psi in MeOH using 5% molar of Pd(OH)₂ for 7 h. The catalyst was filtered, and the filtrate was taken to dryness. This residue was purified by flash chromatography using a DCM/MeOH/NH₄OH mixture (97/2/1) to afford compound 2 as a white solid (93% yield). 1H NMR (CDCl₃): 1.31-1.40 (m, 2H); 1.52-1.58 (m, 2H); 2.10-2.13 (m, 2H); 2.26-2.29 (m, 2H); 2.49 (s, 3H, NCH3); 2.54 (m, 1H); 2.91-3.02 (m, 2H); 3.30 (s, 3H, OCH3); 4.60 (d, 1H, J_{H5-H6}=3.7 Hz); 4.73 (m, 1H); 6.42 (d, 1H, J_{H1-H2}=8.1 Hz; 6.54 (d, 1H, J_{H2-H1}=8.1 Hz); ESI-TOF MS m/z: 317.653 (MH⁺).

OXY(HS).

The amine 2 (80 mg, 0.25 mmol) was dissolved in pyridine, and succinic anhydride (30 mg, 0.3 mmol) was added dropwise. The mixture was refluxed for 7 h. After the disappearance of starting material, the solvent was concentrated to dryness, and the residue was taken up with toluene (three times) and concentrated. This residue was purified by flash chromatography using a DCM/MeOH/NH₄OH mixture (88:10:2) to obtain OXY(HS) as a white solid (82% yield), which was then converted to salt for bioconjugation. 1H NMR (CDCl₃): 1.68-1.77 (m, 2H); 1.79-1.91 (m, 2H); 2.17-2.23 (m, 2H); 2.29 (s, 3H, NCH3); 2.34-2.83 (m, 7H); 3.04 (m, 2H); 3.83 (s, 3H, OMe); 4.09 (m, 1H); 4.63 (d, 1H, J$ 4.7 Hz); 6.33 (d, 1H, J_{H1-H2}=8.2 Hz); 6.37 (d, 1H, J_{H2-H1}=8.2 Hz); 8.03 (d, 1H, J=8.9 Hz, NH); ESI-TOF MS m/z: 417.328 (MH⁺); analytical calculation for C₂₂H₂₈N₂O₆: C, 63.45; H, 6.78; N, 6.73. Found: C, 63.36; H, 6.88; N, 6.72.

Hapten Conjugation.

Haptens were first conjugated to bovine serum albumin (BSA) to optimize reaction efficiency. The difference in molecular weight between the conjugated and unconjugated carrier protein was analyzed by matrix-assisted laser desorption ionization/TOF mass spectrometry and used to calculate the molecular haptenation ratio (number of moles of hapten attached per mole of carrier protein). Haptens were then conjugated to chicken ovalbumin (OVA) for use as coating antigen for ELISA and to KLH for immunization of rats because this protein is acceptable for administration to humans, whereas BSA is not. Hapten (5 mM) and N-ethyl-N′-(3 dimethylaminopropyl)carbodiimide hydrochloride cross-linker (50 mM) were dissolved in 0.1 M MES buffer at pH 4.5. After 10 min of stirring BSA, OVA, or KLH (Thermo Fisher Scientific, Waltham, Mass.) was added at final concentrations of 2.8, 1.9, or 2.8 mg/ml, respectively. These reactant concentrations provided a hapten/protein molar ratio of 120:1 in the reaction mixture for BSA or OVA and produced conjugates with the highest haptenation ratios. Reactions were stirred for 3 h at room temperature and terminated by dialysis in 0.05 M phosphate-buffered saline for 6 h at 4° C. The dialyzed conjugates were sterile-filtered and stored at 4° C. Determination of the haptenation ratio by mass spectrometry was not possible for KLH conjugates because of its large size (˜5 to 8 million Da).

Vaccination.

All protocols were approved by the Minneapolis Medical Research Foundation Animal Care and Use Committee. Male Holtzman rats weighing 350 g (Harlan Teklad, Madison, Wis.) were housed with a 12-h standard light/dark cycle. In immunogenicity, distribution, and behavioral studies, conjugates were injected intraperitoneally in a volume of 0.4 ml, at doses of 25 to 100 μg by using complete Freund's adjuvant for the first injection and incomplete Freund's adjuvant for two subsequent booster injections at 3 and 6 weeks (Pravetoni et al., 2011). A total of three vaccinations were administered, at 0, 21, and 42 days. Immunogenicity, distribution, and behavioral studies were conducted 7 to 10 days after the third immunization. Blood was obtained 7 to 10 days after the last immunization, corresponding to the time of expected peak antibody response, and serum was stored at (20° C.).

ELISA.

ELISA plates were coated with 5 ng/well of OVA conjugate or unconjugated protein control in carbonate buffer at pH 9.6 and blocked with 1% gelatin. OXY(Gly)₄-OVA was used as coating antigen. Primary antibodies were incubated with goat anti-IgG antibodies conjugated to horseradish peroxidase. Antibody specificity was characterized by competitive ELISA, and IC₅₀ values were calculated for those inhibitors that produced distinct plateaus in the maximum percentage of inhibition. Because plateau values differed, IC₅₀ values for each inhibitor were calculated based on the percentage of inhibition of the optical density of primary antibodies incubated with saline. Finally, to establish the relative affinity of anti-OXY antibodies to other structurally related compounds, cross-reactivity percentage was reported.

Effect of Vaccination on OXY Distribution.

Rats were anesthetized with ketamine/xylazine 1 week after the final vaccine dose and an indwelling catheter was placed in their right external jugular vein. Blood was withdrawn for ELISA, and OXY was administered as a 10-s infusion. Rats were decapitated 5 min later, and trunk blood and brain were collected. The effects of immunization with OXY(Gly)₄-BSA or OXY(Gly)₄-KLH doses of 25 and 100 μg were compared in groups of five rats and an OXY dose of 0.5 mg/kg administered intravenously. Controls were vaccinated with unconjugated protein in place of conjugate. The effects of vaccination on the distribution of OXY doses of 0.05, 0.1, and 0.5 mg/kg were compared by using groups of five rats vaccinated with 100 μg of the OXY(Gly)₄-BSA conjugate, 7 to 10 days after the third immunization. The BSA conjugate was used for this experiment because of the ability to verify the haptenation ratio by mass spectrometry. These OXY doses were chosen as representative of the therapeutic single dose range in humans (Leow et al., 1992; Poyhia et al., 1992).

OXY Assay.

Drug doses and concentrations are expressed as weight of the base. OXY concentrations were measured by gas chromatography-MS and represent the total drug (protein or antibody bound as well as free) in each sample. Extraction and quantitation of OXY in brain and serum samples was based on a previously described method (Lewis et al., 2005) using d6-OXY as internal standard (Cerilliant Corporation, Round Rock, Tex.), solid-phase extraction, derivatization with N,O-bis(trimethylsilyl)trifluoroacetamide in 1% trimethylchlorosilane, and analysis on an Agilent 6890 gas chromatograph with methyl siloxane capillary column and 5973 quadrupole mass spectrometer (Agilent Technologies, Santa Clara, Calif.). Adaptations to the method included omission of NaF, analysis of 0.5-ml samples rather than 3-ml samples, and an initial 1:4 rather than 1:2 dilution of brain into 0.1 M phosphate buffer pH 6.0, and solid-phase extraction of brain homogenate supernatant rather than whole homogenate. The limit of quantitation was 5 ng/ml OXY in serum and 50 ng/ml in brain.

Protein Binding.

Equilibrium dialysis was carried out in Sorenson's buffer at pH 7.35 for 4 h at 37° C. by using 1 ml of Teflon cells (Hieda et al., 1997). The free (unbound) oxycodone concentration was calculated as the product of the percentage of bound and the total serum oxycodone concentration before dialysis.

Thermal Nociception Test.

A dose-effect curve was obtained by using groups of six unvaccinated (naive) rats to select an appropriate OXY dose for further study. In addition, serum and brain were obtained from the same naive rats to determine oxycodone concentrations 30 min after subcutaneous dosing. Based on these data, the effects of immunization were tested in rats before and after receiving OXY (2.25 mg/kg s.c.). Groups of 10 rats were vaccinated with 100 μg of OXY(Gly)₄-KLH or unconjugated KLH (controls), and thermal nociception testing was carried out on a hot-plate (Columbus Instruments, Columbus, Ohio) set at 54° C., 7 to 10 days after the third immunization. Rats were habituated to the testing environment for 1 h, and then pretested on the hot-plate to obtain their baseline latency. Two hours later, rats were injected subcutaneously with OXY, and their postdrug latency was obtained 30 min after that. A cutoff value of 60 s was used to prevent tissue damage, and the maximum possible effect (MPE %) was calculated as: (postdrug latency (baseline latency)/(maximal cutoff (baseline latency)×100. Licking the hind paw or jumping were considered endpoints for thermal nociception (Lemberg et al., 2006).

Data Analysis.

Vaccinated groups were compared with unconjugated protein controls by using unpaired two-tailed t tests or, for multiple groups, one-way analysis of variance followed by Bonferroni's post-test. One subject from the OXY(Gly)₄-BSA group and one subject from the KLH group were removed from the analysis of equilibrium dialysis data because the unbound OXY concentrations were <5 ng/ml. The relationship of log titer to brain OXY concentrations was analyzed by linear regression.

Results

Conjugate Vaccine Synthesis.

Conjugation using a molar hapten/protein ratio of 120:1 in the reaction mixture proved optimal and produced BSA conjugates of OXY(Gly)₄ or OXY(HS) with mean haptenation ratios of 16 or 17:1, respectively (Table 1). Hapten was also conjugated to KLH, because KLH is suitable for administration to humans, whereas BSA is not. Conjugation of KLH could not be quantitated by matrix-assisted laser desorption ionization/TOF because of its large molecular weight but was confirmed qualitatively by ELISA in which the conjugate was used as the coating antigen. Conjugates of OXY(Gly)₄-OVA and OXY(HS)-OVA used as the coating ELISA had haptenation ratios of ≧17:1.

Serum Antibody Titers and Affinity.

The OXY(Gly)₄-BSA and OXY(HS)-BSA conjugates had nearly identical haptenation ratios, yet the conjugate containing the (Gly)₄ linker elicited substantially higher titers regardless of the immunogen dose (Table 1). The OXY(Gly)₄-OVA conjugate had a similar haptenation ratio but was not further characterized because it was used only as a coating antigen for ELISA. Competitive ELISA using serum from animals immunized with OXY(Gly)₄-KLH, with OXY(Gly)₄-OVA as the coating antigen (same linker), showed that the resulting antibodies had high affinities (low IC₅₀ values) for both the OXY(Gly)₄ hapten and OXY without linker (Table 2). The (Gly)₄ linker alone showed no measurable competition or binding. Anti-OXY antibodies exhibited 63% cross-reactivity with oxymorphone, a minor but active metabolite of OXY in rat and human (Lalovic et al., 2006; Chan et al., 2008). Affinities for morphine, hydrocodone, hydromorphone, naloxone, and naltrexone were substantially lower, and there was no measurable binding of the off-target opioids methadone or buprenorphine.

OXY Distribution.

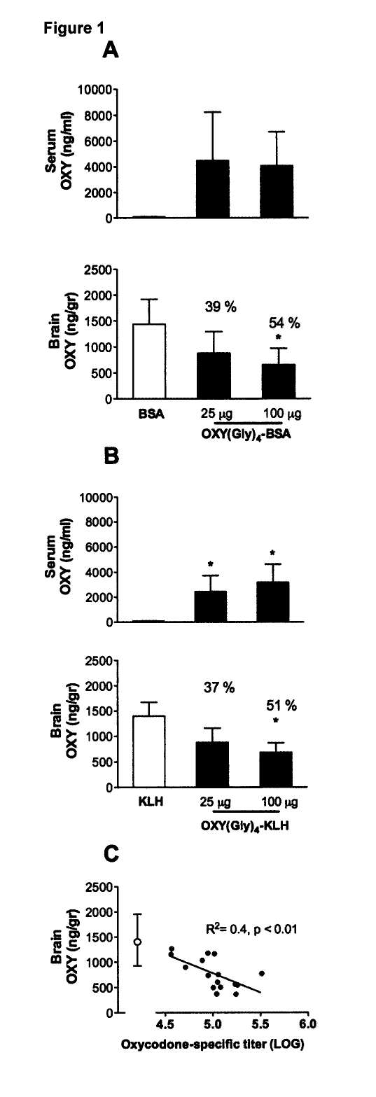

Immunization with OXY(Gly)₄-KLH increased retention of OXY in serum measured 5 min after the OXY dose (FIG. 1) and reduced distribution of OXY to brain. Immunization with OXY(Gly)₄-BSA showed a similar trend toward increased retention of OXY in serum (p=0.07) and, like the KLH conjugate, significantly decreased OXY distribution to brain. There was a trend but no significant difference in efficacy for either immunogen between the 25- and 100-μg doses. However, the 100 μg doses produced reductions of more than 50% in brain OXY concentrations that differed significantly from controls, whereas the 25 μg doses did not, suggesting greater efficacy for the higher immunogen doses (FIG. 1). There was a significant correlation between the log serum antibody titers from all vaccinated rats and their respective brain OXY concentration (FIG. 1).

TABLE 1
Conjugate haptenation ratios and serum antibody titers

| | | Haptenation | Immunogen | Serum Titer |
|---|---|---|---|---|
| Linker | Carrier | Ratio | Dose | (×10³) |
| (Gly)₄ | BSA | 16 | 25 | 80 ± 70 |
| (Gly)₄ | BSA | 16 | 100 | 130 ± 90 |
| (Gly)₄ | KLH | | 25 | 170 ± 110 |
| (Gly)₄ | KLH | | 100 | 110 ± 60 |
| (HS) | BSA | 17 | 100 | 11 ± 11 |
| (HS) | KLH | | 100 | 20 ± 14 |

TABLE 2
Competitive binding IC50 values and cross-reactivity percentage
OXY(Gly)4-OVA was used as ELISA coating antigen.

| Inhibitor | IC₅₀ (μM) | Cross-Reactivity (%) |
|---|---|---|
| OXY(Gly)₄ | 0.003 | >100 |
| OXY | 0.017 | 100 |
| Oxymorphone | 0.03 | 63 |
| Naloxone | 1 | 1.2 |
| Hydromorphone | 4 | 0.4 |
| Naltrexone | 5 | 0.4 |
| Hydrocodone | 10 | 0.2 |
| Morphine | 20 | 0.1 |
| (Gly)₄ | — | |
| Methadone | — | |
| Buprenorphine | — | |
| Nicotine | — | |

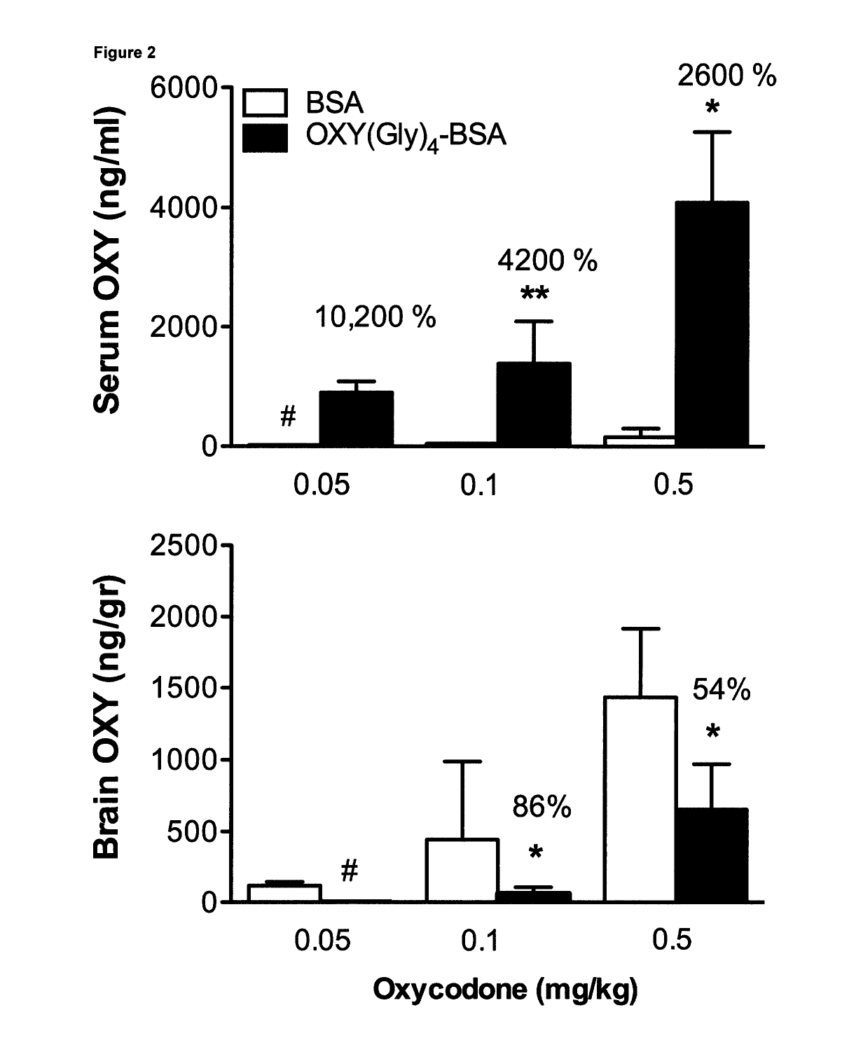

Immunization with 100 μg of OXY(Gly)₄-BSA altered OXY distribution in a dose-related manner (FIG. 2) with substantially larger effects on the lower OXY doses as indicated by the greater percentage of change in serum and brain levels at the lower OXY doses. The distribution of OXY to brain was substantially reduced, by 86% in rats receiving 0.1 mg/kg OXY, and the percentage of reduction could not be calculated at the lowest OXY dose because the brain OXY concentration was below the assay's limit of quantitation.

Protein Binding.

The protein binding of OXY in the serum of control rats was low (10-12%) but increased markedly to 99% in rats vaccinated with either the BSA or KLH conjugate. The free (unbound) OXY concentration in serum was reduced to 19% (BSA conjugate) or 29% (KLH conjugate) that of controls in vaccinated rats (Table 3).

TABLE 3
Oxycodone serum protein binding
Group sizes n = 4 or 5

| | Total OXY | OXY | Unbound OXY |
|---|---|---|---|
| | (ng/ml) | (% bound) | (ng/ml) |
| BSA | 160 ± 150 | 12 ± 2 | 140 ± 130 |
| OXY(Gly)₄-BSA | 3000 ± 1100 *** | 99 ± 1 *** | 30 ± 15 |
| KLH | 80 ± 20 | 10 ± 3 | 70 ± 20 |
| OXY(Gly)₄-KLH | 3200 ± 1500 ** | 99 ± 1 *** | 20 ± 15 * |

* P < 0.05;
** P < 0.01;
*** P < 0.001 compared with controls.

Thermal Nociception Test.

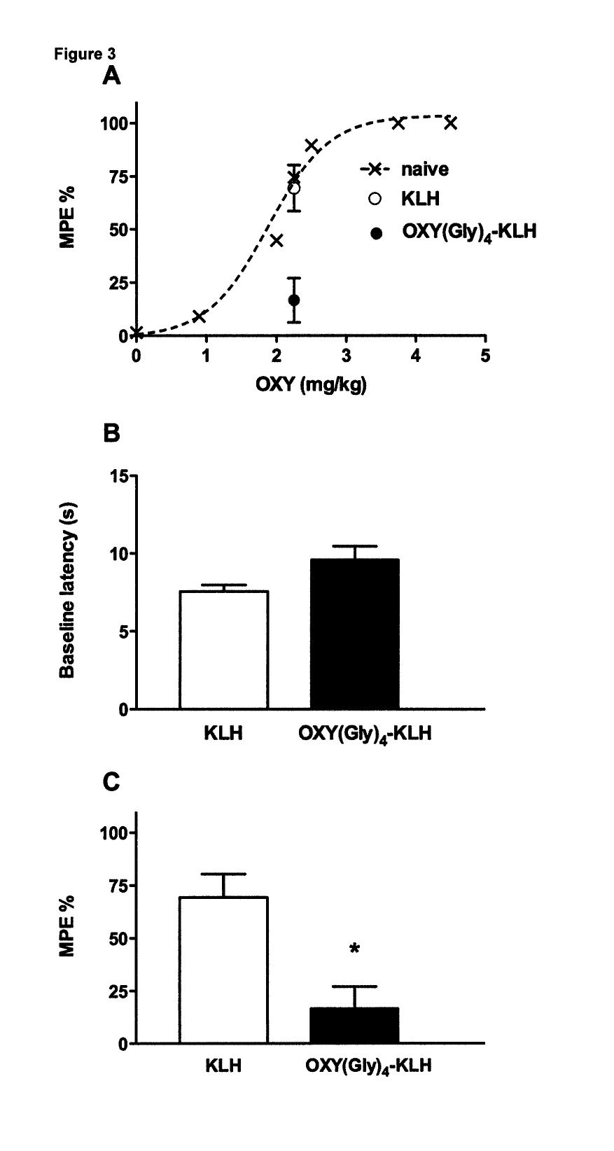

An OXY dose of 2.25 mg/kg was selected for studying effects of immunogens because it produced a nearly maximal effect on latency in unvaccinated naive rats (FIG. 3A). In the same naive rats (n=6) an OXY dose of 2.25 mg/kg, at 30 min after subcutaneous injection, resulted into serum OXY concentrations of 340±50 ng/ml and brain concentrations of 2700±290 ng/ml, indicating that concentrations were higher than concentrations obtained with an intravenous dose of 0.5 mg/kg at 5 min. Baseline latencies did not differ between groups immunized with OXY(Gly)₄-KLH and KLH control (FIG. 3B). The MPE % after OXY administration was significantly lower in the group vaccinated with OXY(Gly)₄-KLH than in the KLH control group (17±10% versus 69±11%; p<0.01) (FIG. 3C).

Discussion

OXY-specific antibodies were highly selective for OXY and its active metabolite oxymorphone and had substantially lower affinities for a variety of off-target opioids. Vaccination of rats with this immunogen increased OXY binding and retention in serum, decreased the free OXY concentration in serum, and decreased OXY distribution to brain after administration of clinically relevant doses of OXY. Vaccination also substantially reduced OXY-induced analgesia in a test of thermal nociception, highlighting its ability to attenuate a centrally mediated behavioral effect of OXY. These data support further study of this immunogen as a potential therapeutic agent for OXY abuse.

Linker composition and length contribute to conjugate vaccine immunogenicity (Kubler-Kielb et al., 2006), but the extent of this contribution is often difficult to interpret because the corresponding haptenation ratios for the conjugates being compared are not reported. Greater conjugate immunogenicity could be caused by enhanced recognition by antigen presenting or B cells but could also be caused by differences in carrier protein haptenation because higher haptenation ratios are generally associated with greater immunogenicity (Carroll et al., 2011). In the current study haptenation ratios for the OXY(Gly)₄-BSA and OXY(HS)-BSA conjugates were nearly identical, yet OXY(Gly)₄-BSA elicited higher ELISA titers. Greater immunogenicity in this case was therefore caused by differences in the linker per se rather than haptenation efficiency. Because (Gly)₄ and HS differ in both composition and length, it is not clear from the available data which of these factors contributed.

Competitive ELISA showed that the full hapten OXY(Gly)₄ had a somewhat higher affinity for vaccine-generated antibodies than did OXY alone. Similar data with a nicotine conjugate vaccine suggest that linkers may contribute to the epitope (part of an antigen) that is recognized by the immune system (Hieda et al., 1997). This is not surprising given the small size of nicotine or OXY. Oligoglycine was used as a linker in this study because it provided a convenient means of studying and optimizing linker length, and (Gly)₄ was shown in preliminary studies to be an effective linker. A potential concern with using a peptide linker is cross-reactivity with native proteins containing (Gly)₄ sequences. This is unlikely because the minimum size generally associated with peptide immunogenicity is 8 to 12 amino acids (Mayrose et al., 2007). In support of this, the (Gly)₄ linker alone produced no measurable inhibition in competitive ELISA. Cross-reactivity of antibodies with native proteins would therefore not be expected.

Antibodies generated by OXY(Gly)₄-KLH cross-reacted with oxymorphone, an active but minor metabolite of OXY in rat and human (Poyhia et al., 1991; Lalovic et al., 2006). It is unlikely that the binding of oxymorphone is necessary for an effective OXY vaccine, because it is largely undetected in human plasma; however, oxymorphone is marketed as an analgesic and its abuse has been reported (http://www.justice. gov/ndic/pubs44/44817/sw0011p.pdf). The OXY(Gly)₄-KLH vaccine could therefore be of interest with regard to oxymorphone abuse. Our findings are similar to a previous report of an OXY immunogen that also used the 6-position for linker attachment and elicited antibodies with similar IC₅₀ values for OXY and oxymorphone (Findlay et al., 1981).

A desirable feature of an OXY vaccine is the production of antibodies that do not bind other opioids that might be needed for therapeutic use in vaccinated individuals. These include methadone, buprenorphine, and naltrexone, which are used for opioid addiction treatment, and naloxone, which is used to reverse excessive opioid effect. Antibodies from the OXY(Gly)₄-KLH vaccine had no measurable affinity for methadone or buprenorphine and had IC50 values substantially higher for naltrexone and naloxone than for OXY. Their clinical use would not be expected to be impeded by vaccination, although this remains to be demonstrated. Cross-reactivity with other abusable opioids could be a useful feature of an OXY vaccine, but this was found only for oxymorphone. Because OXY and oxymorphone differ only in their 3-position substituent, this position does not seem to contribute importantly to the drug-binding epitope. In contrast, absence of a hydroxyl group at the 14-position (hydrocodone and hydromorphone) was sufficient to greatly reduce the affinity for these ligands. These findings help to define the structural features of OXY contributing to epitope recognition.

There are very limited data regarding the effects of opioid vaccines on opioid pharmacokinetics and none pertaining to OXY. Berkowitz and Spector (1972) reported a 7- to 30-fold increase in the serum concentration of [³H]dihydromorphine in mice immunized with a morphine-BSA conjugate vaccine compared with controls and slower radiolabel elimination. Rats or mice passively infused with immune serum from vaccinated rabbits showed reduced distribution of [³H]dihydromorphine to brain (Berkowitz et al., 1974). Hill et al. (1975) found no increase in [¹⁴C]morphine concentration in the serum of rabbits immunized with a similar vaccine but reported modestly prolonged radiolabel elimination. The use of radiolabel rather than specific drug assays is an important limitation of these studies. In the current study, using gas chromatography-mass spectrometry for OXY assay, vaccination with OXY(Gly)₄-BSA greatly enhanced OXY retention in serum and reduced OXY distribution to brain by up to 84%. This magnitude of effect is similar to that of nicotine and cocaine vaccines that block addiction-related behaviors in rats and are in clinical trials (Fox et al., 1996; Pentel et al., 2000). Single oral OXY doses used in humans commonly range from 0.07 to 0.38 mg/kg of immediate release formulations. Corresponding peak or early serum OXY concentrations are typically 10 to 100 ng/ml (Leow et al., 1992; Poyhia et al., 1992). In the current study, OXY was administered over a comparable dose range of 0.05 to 0.5 mg/kg, but intravenously instead of orally, and produced serum OXY concentration sequaling or exceeding the peak levels from oral dosing in humans. Serum OXY concentrations after subcutaneous dosing at 2.25 mg/kg, as used in the hot-plate analgesia experiment, were even higher. Vaccination was effective in altering OXY distribution and hot-plate analgesia despite this rigorous challenge. Greater effects of vaccination on drug distribution at lower OXY doses were anticipated based on data with other addiction vaccines, because vaccine efficacy is greatest when the antibody-to-drug ratio is highest (Keyler et al., 2005).

The large effect of immunization on OXY distribution was mirrored by its behavioral efficacy. Immunization with OXY(Gly)₄-KLH reduced OXY-induced analgesia in a test of thermal nociception, a centrally mediated effect of opioids.

Antibodies are too large to cross the blood-brain barrier, and drug bound to antibody is also excluded from the brain. The primary action of addiction vaccines is presumed to be the binding of drug to antibody in serum, reducing the unbound or free drug concentration and its subsequent distribution to brain (Keyler et al., 2005; Pravetoni et al., 2011). Changes in OXY protein binding and the unbound concentration of OXY in serum after vaccination with OXY(Gly)₄-KLH were substantial and strongly support this mechanism of action. In addition to reducing the early distribution of drug to brain, nicotine vaccines slow nicotine distribution to brain and nicotine elimination (Keyler et al., 1999). These additional effects of vaccination on nicotine disposition may contribute to their effects on drug-related behavior. It will be of interest to examine these parameters for OXY vaccines.

The role of addiction vaccines in treatment is as yet unclear because they are in the early stages of development. It is likely that they will be used as adjunctive therapies or in combination with other medications rather than as single modalities. Antibodies generated by the OXY(Gly)₄-KLH vaccine did not appreciably cross-react with methadone, buprenorphine, or naltrexone so that the therapeutic use of these medications as a treatment for addicts with sufficiently regular and severe opioid use would still be possible. The combination of vaccine and agonist therapy is of interest because a substantial minority of opioid addicts continue to use their opioid of choice even while on methadone maintenance therapy (D'Aunno and Vaughn, 1992). It is of course possible that someone abusing OXY, the opioid targeted by this vaccine, could switch to abusing a different opioid. Combining a vaccine with other adjunctive therapies would presumably help to minimize this possibility. It may also be feasible to combine multiple vaccines directed at different opioids to provide broader coverage. This strategy is widely used for infectious diseases by combining multiple unrelated vaccines, e.g., measles, mumps, and rubella for convenience. Because the immune system has the capacity to respond to multiple simultaneous challenges, the individual vaccine components retain their individual immunogenicity when combined in this manner. Early data with nicotine vaccines suggest that this strategy can be generalized to conjugate vaccines as well (Keyler et al., 2008). Whichever approach is adopted, the development of an effective OXY vaccine represents a first step toward investigating whether this is a viable therapeutic strategy.

### Example 1 References

Anton B and Leff P (2006) A novel bivalent morphine/heroin vaccine that prevents relapse to heroin addiction in rodents. Vaccine 24:3232-3240.
Anton B, Salazar A, Flores A, Matus M, Marin R, Hernandez J A, and Leff P (2009) Vaccines against morphine/heroin and its use as effective medication for preventing relapse to opioid addictive behaviors. Hum Vaccin 5:214-229.
Berkowitz B and Spector S (1972) Evidence for active immunity to morphine in mice. Science 178:1290-1292.
Berkowitz B A, Ceretta K V, and Spector S (1974) Influence of active and passive immunity on the disposition of dihydromorphine-H3. Life Sci 15:1017-1028.
Bieniarz C, Barnes G, Welch C J, and Schlesinger C A (1988) inventors; Abbott Laboratories, assignee. Preparation of poly(amino acid)-based heterobifunctional coupling agents and conjugates containing them. European patent application 314127 A2 19890503. 1988 Oct. 27.
Bonese K F, Wainer B H, Fitch F W, Rothberg R M, and Schuster C R (1974) Changesin heroin self-administration by a rhesus monkey after morphine immunisation. Nature 252:708-710.
Carroll F I, Blough B E, Pidaparthi R R, Abraham P, Gong P K, Deng L, Huang X, Gunnell M, Lay J O Jr, Peterson E C, et al. (2011) Synthesis of mercapto-(%)-methamphetamine haptens and their use for obtaining improved epitope density on (%)-methamphetamine conjugate vaccines. J Med Chem 54:5221-5228.
Chan S, Edwards S R, Wyse B D, and Smith M T (2008) Sex differences in the pharmacokinetics, oxidative metabolism and oral bioavailability of oxycodone in the Sprague-Dawley rat. Clin Exp Pharmacol Physiol 35:295-302.
Chi K R (2011) Vaccines move forward against a range of addictions. Nat Med 17:146. Compton W M and Volkow N D (2006) Major increases in opioid analgesic abuse in the United States: concerns and strategies. Drug Alcohol Depend 81:103-107.
D'Aunno T and Vaughn T E (1992) Variations in methadone treatment practices. Results from a national study. JAMA 267:253-258.
Dodrill C L, Helmer D A, and Kosten T R (2011) Prescription pain medication dependence. Am J Psychiatry 168:466-471.
Fareed A, Vayalapalli S, Stout S, Casarella J, Drexler K, and Bailey S P (2011) Effect of methadone maintenance treatment on heroin craving, a literature review. J Addict Dis 30:27-38.
Findlay J W, Butz R F, and Jones E C (1981) Relationships between immunogen structure and antisera specificity in the narcotic alkaloid series. Clin Chem 27: 1524-1535.
Fox B S, Kantak K M, Edwards M A, Black K M, Bollinger B K, Botka A J, French T L, Thompson T L, Schad V C, Greenstein J L, et al. (1996) Efficacy of a therapeutic cocaine vaccine in rodent models. Nat Med 2:1129-1132.
Hatsukami D K, Jorenby D E, Gonzales D, Rigotti N A, Glover E D, Oncken C A, Tashkin D P, Reus V I, Akhavain R C, Fahim R E, et al. (2011) Immunogenicity and smoking-cessation outcomes for a novel nicotine immunotherapeutic. Clin Pharmacol Ther 89:392-399.
Hieda Y, Keyler D E, Vandevoort J T, Kane J K, Ross C A, Raphael D E, Niedbalas R S, and Pentel P R (1997) Active immunization alters the plasma nicotine concentration in rats. J Pharmacol Exp Ther 283:1076-1081.
Hill J H, Wainer B H, Fitch F W, and Rothberg R M (1975) Delayed clearance of morphine from the circulation of rabbits immunized with morphine-6-hemisuccinate bovine serum albumin. J Immunol 114:1363-1368.
Keyler D E, Hieda Y, St Peter J, and Pentel P R (1999) Altered disposition of repeated nicotine doses in rats immunized against nicotine. Nicotine Tob Res 1:241-249.
Keyler D E, Roiko S A, Benlhabib E, LeSage M G, St Peter J V, Stewart S, Fuller S, Le C T, and Pentel P R (2005) Monoclonal nicotine-specific antibodies reduce nicotine distribution to brain in rats: dose- and affinity-response relationships. Drug Metab Dispos 33:1056-1061.
Keyler D E, Roiko S A, Earley C A, Murtaugh M P, and Pentel P R (2008) Enhanced immunogenicity of a bivalent nicotine vaccine. Int Immunopharmacol 8:1589-1594.
Kubler-Kielb J, Liu T Y, Mocca C, Majadly F, Robbins J B, and Schneerson R (2006) Additional conjugation methods and immunogenicity of Bacillus anthracis poly-,-D-glutamic acid-protein conjugates. Infect Immun 74:4744-4749.
Lalovic B, Kharasch E, Hoffer C, Risler L, Liu-Chen L Y, and Shen D D (2006) Pharmacokinetics and pharmacodynamics of oral oxycodone in healthy human subjects: role of circulating active metabolites. Clin Pharmacol Ther 79:461-479.
Lamont J V, McConnell R I, Fitzgerald S P, Benchikh E O, and Lowry A P (2003) inventors; Randox Laboratories Ltd., assignee. Preparation of haptens, immunogens and antibodies to oxycodone and its metabolites. European Patent Application 1323718 A2 20030702.2003 Aug. 21.
Lemberg K, Kontinen V K, Viljakka K, Kyla\ddot{}nlahti I, Yli-Kauhaluoma J, and Kalso E (2006) Morphine, oxycodone, methadone and its enantiomers in different models of nociception in the rat. Anesth Analg 102:1768-1774.
Leow K P, Smith M T, Watt J A, Williams B E, and Cramond T (1992) Comparative oxycodone pharmacokinetics in humans after intravenous, oral, and rectal administration. Ther Drug Monit 14:479-484.
Lewis R J, Johnson R D, and Hattrup R A (2005) Simultaneous analysis of thebaine, 6-MAM and six abused opioids in postmortem fluids and tissues using Zymark automated solid-phase extraction and gas chromatography-mass spectrometry. J Chromatogr B Analyt Technol Biomed Life Sci 822:137-145.
Lopez M F, Compton W M, and Volkow N D (2009) Changes in cigarette and illicit drug use among U S teenagers. Arch Pediatr Adolesc Med 163:869-870.
Martell B A, Orson F M, Poling J, Mitchell E, Rossen R D, Gardner T, and Kosten T R (2009) Cocaine vaccine for the treatment of cocaine dependence in methadone maintained patients: a randomized, double-blind, placebo-controlled efficacy trial. Arch Gen Psychiatry 66:1116-1123.
Maxwell J C (2011) The prescription drug epidemic in the United States: a perfect storm. Drug Alcohol Rev 30:264-270. Mayrose I, Shlomi T, Rubinstein N D, Gershoni J M, Ruppin E, Sharan R, and Pupko T (2007) Epitope mapping using combinatorial phage-display libraries: a graphbased algorithm. Nucleic Acids Res 35:69-78.
Pentel P R, Malin D H, Ennifar S, Hieda Y, Keyler D E, Lake J R, Milstein J R, Basham L E, Coy R T, Moon J W, et al. (2000) A nicotine conjugate vaccine reduces nicotine distribution to brain and attenuates its behavioral and cardiovascular effects in rats. Pharmacol Biochem Behav 65:191-198.
Po\ddot{}yhia\ddot{}R, Olkkola K T, Seppa\ddot{}la\ddot{} T, and Kalso E (1991) The pharmacokinetics of oxycodone after intravenous injection in adults. Br J Clin Pharmacol 32:516-518.
Po\ddot{}yhia\ddot{}R, Seppa\ddot{}la\ddot{} T, Olkkola K T, and Kalso E (1992) The pharmacokinetics and metabolism of oxycodone after intramuscular and oral administration to healthy subjects. Br J Clin Pharmacol 33:617-621.
Pravetoni M, Keyler D E, Raleigh M D, Harris A C, Lesage M G, Mattson C K, Pettersson S, and Pentel P R (2011) Vaccination against nicotine alters the distribution of nicotine delivered via cigarette smoke inhalation to rats. Biochem Pharmacol 81:1164-1170.
Sayre L M and Portoghese P S (1980) Stereospecific synthesis of the 6--- and 6-.-amino derivatives of naltrexone and oxymorphone. J Org Chem 45:3366-3368.
Stotts A L, Dodrill C L, and Kosten T R (2009) Opioid dependence treatment: optionsin pharmacotherapy. Expert Opin Pharmacother 10:1727-1740.
Stowe G N, Vendruscolo L F, Edwards S, Schlosburg J E, Misra K K, Schulteis G, ayorov A V, Zakhari J S, Koob G F, and Janda K D (2011) A vaccine strategy that induces protective immunity against heroin. J Med Chem 54:5195-5204.
Wainer B H, Fitch F W, Rothberg R M, and Fried J (1972) Morphine-3-succinyl-bovine serum albumin: an immunogenic hapten-protein conjugate. Science 176:1143-1145.
Wainer B H, Fitch F W, Rothberg R M, and Schuster C R (1973) In vitro morphine antagonism by antibodies. Nature 241:537-538.

### Example 2

### Co-Administration of Morphine and Oxycodone Vaccines Reduces the Distribution of 6-monoacetylmorphine and Oxycodone to Brain in Rats

Opioid abuse and addiction in the USA encompasses a wide variety of opioids [1,2]. Prior to the 1990s, heroin abuse was predominant and was the focus of treatment strategies. Over the past 15 years prescription opioid abuse has increased dramatically and is now substantially more common than that of heroin [3,4]. Oxycodone and to a lesser extent hydrocodone or oxymorphone abuse have been increasingly reported in various populations, including teens and USA military personnel [1,5-7]. Patterns of opioid abuse are also diverse. Daily use by intravenous injection or smoke inhalation is common with heroin, while more occasional oral or intravenous use is more common with prescription opioids [8]. Existing medications for the treatment of opioid addiction are effective and helpful, yet are taken advantage of by only a small fraction of opioid abusers [9]. Agonist therapies including methadone and buprenorphine are themselves addictive. Their use requires substantial oversight, and is legally restricted to established daily opioid users. Despite the potential benefits, some opioid addicts object to taking an addictive treatment medication. Because abuse of prescription opioids is often episodic, continuous agonist therapy is a less attractive option to treat this pattern of abuse. The opioid antagonist naltrexone is effective for heroin addiction, but compliance is generally poor. Additional treatment options are needed which address the diversity of both the opioids abused and their different routes of administration and patterns of use [10].

Vaccines targeting drugs of abuse are being developed as an alternative or supplemental approach to addressing addictions [11]. These vaccines stimulate production of antibodies, which bind the target drug, alter its distribution to brain and reduce drug related behaviors in animals. Vaccines against cocaine and nicotine have reached clinical trials [12,13]. A number of vaccines have been developed, which elicit antibodies that bind heroin and its sequentially produced active metabolites 6-monoacetylmorphine (6-MAM), morphine, and morphine-6-glucuronide. Some of these vaccines have been shown to reduce heroin- or morphine-induced behaviors, including self-administration in animals [14-19]. These vaccines show structural specificity and little binding of other opioids such as methadone, buprenorphine, or naltrexone. This structural specificity is advantageous in that use of such vaccines should not preclude the concurrent use of agonist therapies, but high specificity also means that these vaccines do not appreciably bind other abusable opioids such as oxycodone. An oxycodone vaccine was recently described, which binds oxycodone and its active metabolite oxymorphone but has a much lower affinity for heroin and its metabolites, reduces oxycodone distribution to brain and reduces oxycodone-induced hot plate analgesia in rats [20]. The availability of this vaccine presents the possibility of combining heroin and oxycodone vaccines in order to achieve broader opioid binding activity.

In the current study rats received an oxycodone-KLH conjugate vaccine (OXY-KLH) targeting oxycodone and its active metabolite oxymorphone, or a morphine-KLH conjugate vaccine (M-KLH) targeting heroin, 6-MAM and morphine. The two vaccines were administered alone or in combination to determine whether their combined use would preserve their individual efficacies. Because heroin is rapidly converted in vivo to 6-MAM which is considered largely responsible for its acute effects, and resulting heroin levels are quite low [21], 6-MAM was used as a model opioid in this study rather than heroin. Rats vaccinated with M-KLH and OXYKLH vaccines concurrently developed high titers of antibodies to all of the targeted drugs and showed substantial reductions in the distribution of 6-MAM and oxycodone to brain.

2. Material and Methods

2.1. Drugs and Reagents

6-monoacetylmorphine (6-MAM), morphine, oxycodone and opioids used for competitive binding studies were obtained through the NIDA Drug Supply Program and Sigma (St. Louis, Mo.). [Leu5]-enkephalin was purchased from Tocris Biosciences (Ellisville, Mo.). All drug doses and concentrations are expressed as the weight of the base.

2.2. Synthesis of Haptens

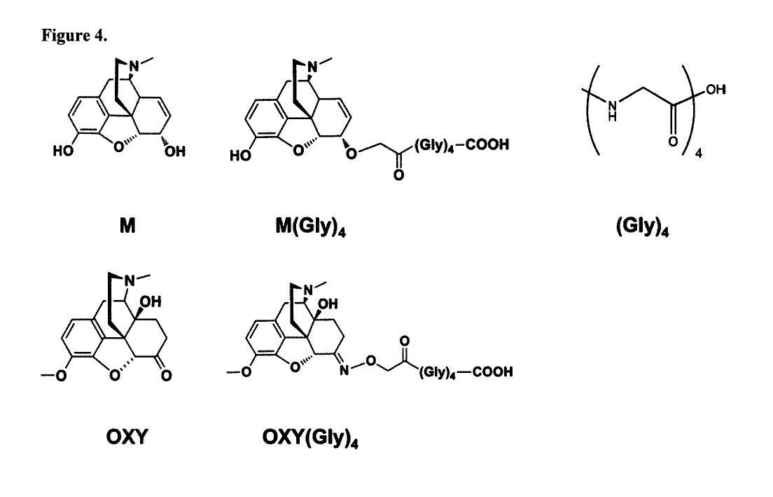

OXY(Gly)₄ hapten consisting of oxycodone with a tetraglycine (Gly)₄ linker at the C6 position was synthesized as previously described (FIG. 4) [20].

The analogous morphine M(Gly)₄ hapten with C6 linker position was synthesized as detailed below. This linker position was selected because of its efficacy in previously reported immunogens for eliciting antibodies against heroin, 6-MAM, morphine and morphine-6-glucoronide[14,18], and its efficacy for the OXY-KLH vaccine [20]. The (Gly)₄ linker length was found to be more effective than shorter linkers for the OXY-KLH vaccine and is similar to the linker length of an heroin vaccine shown to be highly immunogenic and effective in blocking heroin-induced behaviors [14]. For characterization of synthetic intermediates and haptens, ¹H and ¹³C nuclear magnetic resonance (NMR) spectra were taken on a Bruker Avance 400 MHz instrument (Brucker, Billerica, Mass.) and calibrated using an internal reference. Elemental analyses were performed by M-H-W Laboratories (Phoenix, Ariz.). Analytical thin-layer chromatography (TLC) was performed on EM Science (Gibbstown, N.J.) silica gel 60 F254 (0.25 mm) and plates visualized by UV light, iodine vapor, or ninhydrine solution. 3-Boc-M (1): Morphine sulfate (5 mmol) was dissolved in a dimethylformamide (DMF)/H2O mixture (9/1: v/v) and catalytic 4-dimethylaminopyridine (DMAP) (0.025 mmol) was added. Tert-Butyloxycarbonyl (Boc) anhydride (1.1 equivalent) dissolved in DMF was added over 30 min and the mixture stirred at room temperature overnight. Solvents were evaporated under reduced pressure and the residue was purified by flash chromatography (3% MeOH/DCM/1% NH4OH) on silica gel (230-400 mesh, Fisher Scientific, Pittsburgh, Pa.) to afford 1 (84% yield).

3-BOC-M-6-O-tertButylglycolate (2): A suspension of sodium hydride (60% dispersion in mineral oil) (2.9 mmol) in dry tetrahydrofuran (5 ml) at 0° C. under nitrogen was gradually added to 1 (1.9 mmol) and the reaction mixture was allowed to stir at room temperature for 30 min, followed by the addition of tertbutylbromoacetate (2.9 mmol). The reaction mixture was stirred at room temperature for 8 h. The reaction was concentrated under vacuum, diluted with water and then extracted with dichloromethane (DCM). The organic layers were combined, dried over sodium sulfate, filtered and concentrated under vacuum to furnish a final crude residue. This residue was subjected to flash column chromatography using a DCM/MeOH/NH₄OH mixture (97/2/1) to afford 2 as an oil (67% yield). M-6-O-glycolic acid (3): Trifluoroacetic acid (TFA) (20% v/v) was added to a solution of the ester 2 (2 mmol) in DCM (20 ml). The resultant solution was stirred at room temperature. Upon complete disappearance of starting material, the solvent was removed under vacuum. The crude reaction mixture was subjected to azeotropic drying using toluene. The residue was taken up in a methanol/diethyl ether (Et₂O) mixture and the precipitate was filtered and washed with Et2O to afford 3 as a white solid (85% yield). M(Gly)₄: The M-6-O-glycolic acid (3) was coupled to tetraglycine tertbutyl esters (Gly4tBu) using Dicyclohexylcarbodiimide (DCC)/hydroxybenzotriazole (HOBt) followed by acid hydrolysis to complete the synthesis of M(Gly)₄ as described before for OXY(Gly)_{4 [}20]. M-6-O-glycolic acid (3) (0.15-0.4 mmol), DCC (1.3 equivalent) and HOBt (1.2 equivalent) were dissolved in 5 ml of anhydrous DMF. The solution was cooled to 0° C., placed under a nitrogen atmosphere, and after 15 min Gly₄tBu (0.15-0.4 mmol) was added. The solution was sealed under a nitrogen atmosphere and was allowed to reach room temperature and then stir overnight. The reaction mixture was filtered to remove dicyclohexylurea into water (10× initial volume of DMF) and extracted with ethylacetate. The combined organic layers were dried on magnesium sulfate, and concentrated under reduced pressure. The residue was purified by flash column chromatography (DCM/MeOH/NH₄OH: 94/5/1) and to a solution of this protected intermediate (0.15-0.4 mmol scale) in DCM (5 ml) was added TFA (20% v/v). The resultant solution was stirred at room temperature. Upon complete disappearance of starting material, the solvent was removed under vacuum. The crude reaction mixture was subjected to azeotropic drying using toluene. The residue was taken up again in a methanol/Et₂O mixture and the precipitate was filtered and washed with Et₂O. The final crude was purified by reverse phase high pressure liquid chromatography using an acetonitrile/H2O/TFA (80/20/0.1%) mixture to provide M(Gly)₄ as a slightly yellow solid (43% over 2 steps). M(Gly)₄ was converted to its TFA salt for conjugation to carrier proteins.

2.3. Conjugation and Purification of Conjugates

Haptens for use in the vaccines were conjugated to keyhole limpet hemocyanin (KLH) because this protein is highly immunogenic and acceptable for use in humans. For use as coating antigen in ELISA assays, haptens were conjugated to bovine serum albumin (BSA) or chicken albumin (OVA). Hapten 5 mM and ethyl-N′-(3 dimethylaminopropyl)carbodiimide hydrochloride 50 mM were dissolved in 0.1 M MES buffer at pH 4.5. BSA, OVA or KLH (Thermo Fisher Rockford, Ill.) were added and reactions were stirred for 3 h at room temperature followed by dialysis and storage at +4° C. as previously described [20]. Molar hapten:protein conjugation ratios (moles of hapten conjugated per mole of protein) for the BSA and OVA conjugates were quantitated by MALDI-TOF (Reflex III, Bruker).

2.4. Vaccination

All animal studies have been carried out in accordance with EU Directive 2010/63/EU and approved by the Minneapolis Medical Research Foundation Animal Care and Use Committee. Male Holtzman rats weighing 350 g (Harlan Laboratories, Madison, Wis.) were housed with a 12/12 h standard light/dark cycle. Conjugates were injected i.p. in a final volume of 0.4 ml in complete Freund's adjuvant for the first injection and incomplete Freund's adjuvant for 2 subsequent booster injections at 3 and 6 weeks. Experiments were conducted 7-10 days after the 3rd immunization. Rats (n=12 group) were immunized as follows, with unconjugated KLH added as needed so that all groups received a total KLH protein dose of 50 μg: Group (1) KLH 50 μg; Group (2) Monovalent M-KLH 25 μg+KLH 25 μg; Group (3) Monovalent OXY-KLH 25 μg+25 μg KLH; Group (4) Bivalent M-KLH 25 μg+OXY-KLH 25 μg.

To compare vaccine route and adjuvant male BALB/c mice (n=5 per group) (Harlan Laboratories, Madison, Wis.) were vaccinated on days 0, 14 and 28 with 25 μg of monovalent OXY-KLH. Vaccine was administered either i.p. in a final volume of 0.2 ml in complete Freund's adjuvant for the first dose and incomplete Freund's for the remaining doses, or s.c. in a final volume of 0.2 ml containing alum adjuvant (Thermo Fisher) at a final concentration of 5 mg/ml. Blood was obtained on day 35 for serum antibody titer measurement.

2.5. Antibody Cross-Reactivity and Specificity

ELISA plates were coated with 5 ng/well of M-BSA or OXY-OVA conjugate or unconjugated protein control in carbonate buffer at pH 9.6 and blocked with 1% gelatin. Rat primary antibodies were incubated with goat anti-IgG antibodies conjugated to horseradish peroxidase, while rabbit anti-mouse IgG antibodies were used to measure mouse immunized serum. The extent of cross-reactivity to immunogen between antibodies generated by each monovalent vaccine was assessed by measuring titers against both the M-BSA and OXY-OVA coating immunogens. Cross-reactivity for the M-KLH vaccine was calculated as the ratio of its ELISA titer to OXY-OVA divided by its ELISA titer to M-BSA and vice versa for the OXYKLH vaccine. Antibody specificity was characterized by competitive ELISA and IC₅₀ values for each inhibitor were obtained as described before [20].

2.6. Morphine-Specific Antibody Concentration

Morphine-specific IgG concentrations from immunized rats were measured by ELISA using a standard curve constructed using commercially available murine monoclonal anti-morphine IgG. The morphine-conjugate used to generate the monoclonal antimorphine IgG utilized the same C6 linker position for conjugation to carrier protein (BSA) as the M(Gly)₄ immunogen used in the current study (Qed Biosciences, San Diego, Calif.). A corresponding oxycodone monoclonal antibody with C6 linker position was not available so this approach could not be used to measure anti-oxycodone IgG concentrations. Because the serum morphine-specific antibody being measured was from rats while the monoclonal IgG used for the standard curve was from mice, preliminary experiments were performed to determine dilutions of anti-mouse IgG-horseradish peroxidase (HRP) or anti-rat IgG-HRP, which produced the same optical density reading when added to wells containing 5 ng of commercially obtained purified mouse or rat IgG, respectively. These secondary antibody dilutions were then used for the standard curve or serum ELISAs to quantitate morphine-specific IgG levels in vaccinated rats. For analysis of rat serum, wells were coated with 5 ng of M-BSA and serum dilutions were added followed by goat anti-rat IgG-HRP at a 1:50,000 dilution. The corresponding standard curve was obtained by coating ELISA wells similarly and adding various concentrations of anti-morphine monoclonal antibody followed by rabbit antimouse IgG-HRP at a 1:10,000 dilution. Controls consisting of wells coated with BSA alone showed no binding of either the monoclonal antibody or immune serum to this protein.

2.7. Effects of Vaccination on 6-MAM, Morphine and Oxycodone

Distribution. The effects of immunization with the bivalent M-KLH+OXYKLH vaccine on drug distribution were measured in rats receiving 0.1 mg/kg (0.32 μmol/kg) 6-MAM and the same dose of oxycodone administered concurrently i.v. 6-MAM was dissolved in 2% (v/v) DMSO in physiological saline while oxycodone was dissolved directly in saline on the day of the experiment. 6-MAM and oxycodone were mixed 1:1 (v:v) in the same syringe prior to intravenous infusion. One week after the final vaccine dose rats were anesthetized with ketamine/xylazine (respectively 100 mg/kg and 10 mg/kg) and an indwelling catheter was placed in their right external jugular vein. Blood was withdrawn for ELISA assays, and 6-MAM+oxycodone were administered as a 60 s infusion. Rats were decapitated 4 min later and trunk blood and brain collected. Oxycodone and 6-MAM doses were chosen as the largest doses that, when combined, would avoid respiratory depression or overdose.

2.8. Oxycodone Assay

Serum and brain oxycodone or oxymorphone concentrations were measured by gas chromatography coupled to mass spectrometry as previously described [20]. The reported oxycodone concentrations represent the total drug (protein or antibody-bound as well as free) in each sample.

2.9. 6-MAM and Morphine Assay

All solutions and samples were kept on ice. Whole blood samples were collected into a syringe containing ice-cold sodium fluoride (4 mg/ml) and heparin (100 IU/ml). Plasma was separated and 0.5 ml of plasma was diluted 1:1 with formate buffer (pH 3.0, 10 mM). Brain halves were rinsed with 10 mM formate (pH 3.0), placed into pre-weighed vials, then weighed and 10 mM formate pH 3.0 was added (4 parts by weight). Samples were homogenized 30-40 s and stored at −20° C. until extraction. Samples underwent solid-phase extraction using Strata-X-RP 1 ml extraction cartridges and were quantified by liquid chromatography coupled to mass spectrometry (LC-MS). All samples were extracted immediately after sample collection to avoid degradation of heroin and 6-MAM. Standard curves were prepared using bovine serum and were linear in the range of 5-200 ng/ml for 6-MAM and 10-200 ng/ml for morphine. The internal standards were the deuterated analogues of 6-MAM and morphine (Cerilliant Analytical Reference Standards, Round Rock, Tex.). Samples were analyzed on a 2010A Shimadzu (Tokyo, Japan) single-quadrupole LC-MS with reversed phase Agilent (Santa Clara, Calif.) Zorbax XDB-C18 (2.1 mm×50 mm i.d., 3.5 μm) column with a reversed phase 4 mm×2 mm C18 guard column (Phenomenex, Torrance, Calif.) and a gradient mobile phase mixture of 10 mM formate buffer pH 3.0 and acetonitrile. Total run time was 5 min. Validation was based on a six point standard curve prepared in triplicate (within-run) for each day on five different days (between run, N=15). The within day variability was between 2.2% and 7.9%, the between day variability 1.9% and 6.9%, and the limit of quantitation 5 ng/ml.

2.10. Stoichiometric Relationships

The total number of moles/kg of anti-morphine IgG elicited in rats vaccinated with M-KLH was calculated as the product of the mean serum anti-morphine IgG concentration and its reported volume of distribution of 131 ml/kg in rats [22]. The corresponding number of drug-binding sites of IgG was twice that number since there are 2 binding sites per IgG. The % saturation of anti-morphine IgG in serum was calculated as the molar ratio of the antibody bound serum ligand concentration (6-MAM+morphine) to the serum anti-morphine IgG binding site concentration. Antibody bound drug concentration in serum was calculated as (total 6-MAM+morphine concentration in the M-KLH group) minus (mean 6-MAM+morphine concentration in the control KLH group).

The drug assay used measured total (bound and unbound) drug in each sample so that the total concentration in the M-KLH group represented both bound and unbound drug, while the concentrations in the control group represented only unbound drug since there are not antibodies present. The difference between these therefore provided an estimate of the bound drug concentration.

TABLE 4
Serum antibody titers ×10³ and cross-reactivity (%)^{a}.

| | Vaccine | | |
|---|---|---|---|
| | | | M-KLH + |
| Coating immunogen | M-KLH | OXY-KLH | OXY-KLH |
| M-BSA | 140 ± 47 | 18 ± 14 | 325 ± 220* |
| OXY-OVA | 29 ± 17 (21%) | 102 ± 78 | 239 ± 172* |

^{a}Cross-reactivity for each monovalent vaccine was calculated as the ratio of titer against the non-target immunogen/target immunogen.
*p < 0.05 v. monovalent vaccine.

2.11. Statistical Analysis and Calculations

Group differences were analyzed by one-way analysis of variance followed by Bonferroni post hoc test. Relationships between serum antibody titers or concentrations and drug concentrations in serum and brain were analyzed by linear regression within groups and analysis of covariance was used to compare their linear regression slopes across groups.

3. Results

3.1. Synthesis and Conjugation of M-KLH and OXY-KLH Vaccines

Haptenation ratios for M(Gly)₄ or OXY(Gly)₄ conjugated to BSA or OVA ranged from 17 to 21 mol of hapten per mole of carrier protein. Haptenation ratios for the M-KLH and OXY-KLH conjugates could not be measured by mass spectrometry due to the larger size of KLH.

3.2. Antibody Response in Rats

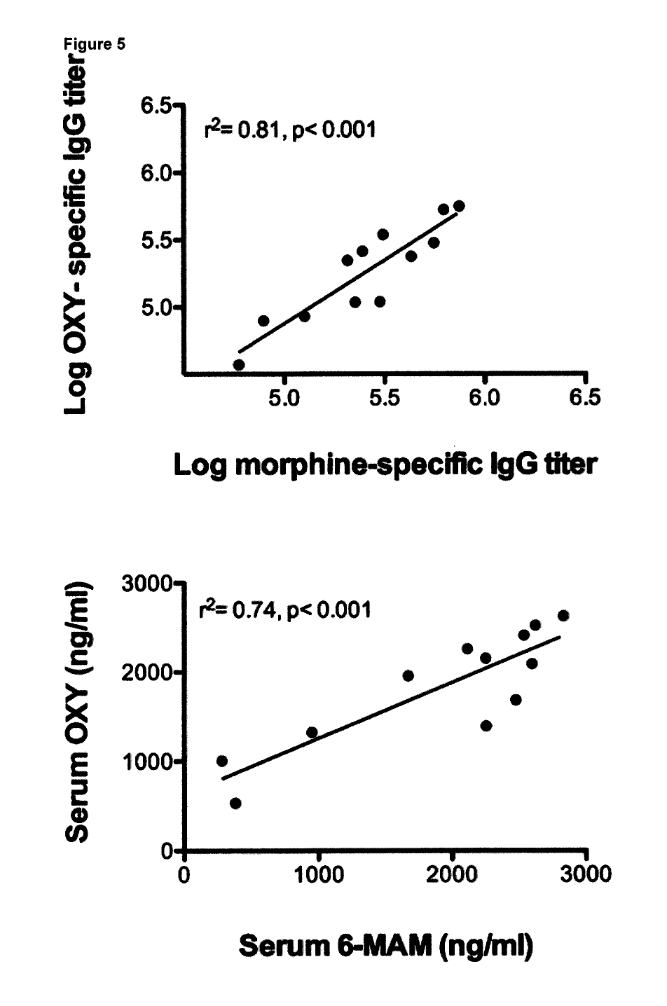

Monovalent M-KLH: Immunization with M-KLH alone elicited serum antibodies that were highly specific for heroin, 6-MAM and morphine. Serum from rats vaccinated with M-KLH showed 20% cross-reactivity with the OXY-OVA coating antigen on ELISA, and negligible cross-reactivity with unconjugated OVA alone (Table 4). Competition ELISA showed high relative affinities of antiserum (low IC50 values) for heroin, morphine and 6-MAM, and a low relative affinity for oxycodone. Cross-reactivity with the (Gly)₄ linker alone was negligible. Serum anti-morphine IgG concentrations were consistently high, with a mean±SD of 558±62 μg/ml and no values below 452 μg/ml. Monovalent OXY-KLH: Immunization with OXY-KLH alone elicited serum antibodies that were highly specific for oxycodone and oxymorphone. Serum from rats vaccinated with OXY-KLH showed 18% cross-reactivity with the M-BSA coating antigen on ELISA and negligible cross-reactivity with unconjugated BSA alone (Table 4). Competition ELISA values showed high relative affinities for oxycodone and oxymorphone, low relative affinity for heroin, 6-MAM or morphine, and negligible cross-reactivity with the (Gly)₄ linker alone or the endogenous opioid Leu-enkephalin similarly to our previous report [20]. Bivalent M-KLH and OXY-KLH: Rats immunized with the bivalent vaccine developed serum antibody titers against each immunogen that were higher than in rats vaccinated with monovalent vaccines alone (Table 4). These titers were greater than could be accounted for simply by adding the increase in titers attributable to cross-reactivity of the antibodies as determined by ELISA. For example, the anti-morphine titer in the bivalent group of 325×10³ is greater than the anti-morphine titer of 140×10³ in the group vaccinated with monovalent M-KLH even allowing for an additional titer of 29×10³, which would be expected in the bivalent group due to cross-reactivity from immunization with OXY-KLH. Antibody specificities in the bivalent group, as determined by IC₅₀ values, were similar to those obtained with the monovalent vaccines. While there was considerable individual variability in serum antibody titers in the bivalent group, there was a high correlation between anti-morphine and anti-oxycodone antibody titers measured in individual animals (FIG. 5).

3.3. Antibody Response in Mice

Monovalent OXY-KLH vaccine produced comparable antibody titers in BALB/c mice whether injected i.p. in Freund's adjuvant (43±14×10³, mean±SE) or s.c. in alum adjuvant (44±16×10³)(p>0.05).

3.4. Effects of Vaccination on Drug Distribution

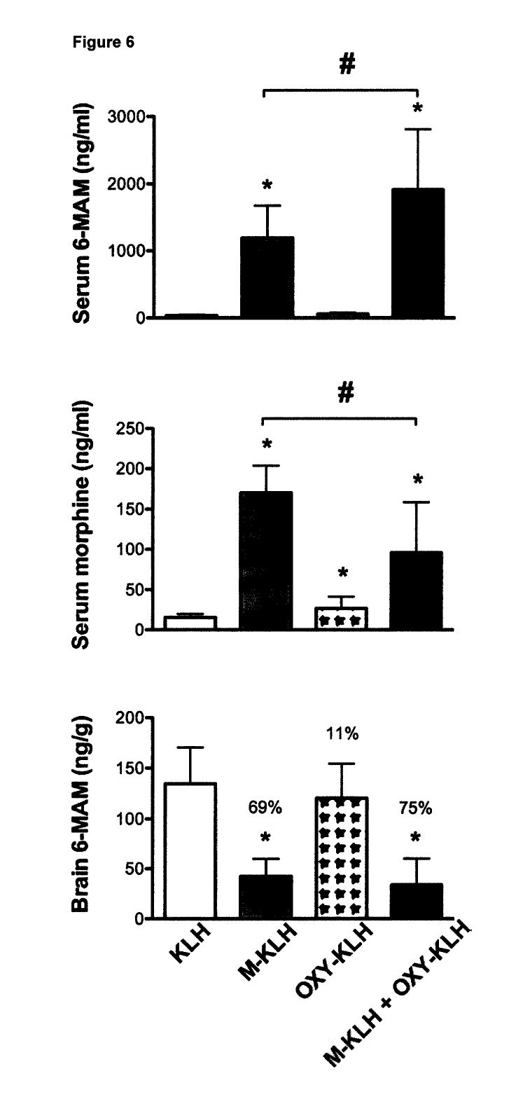

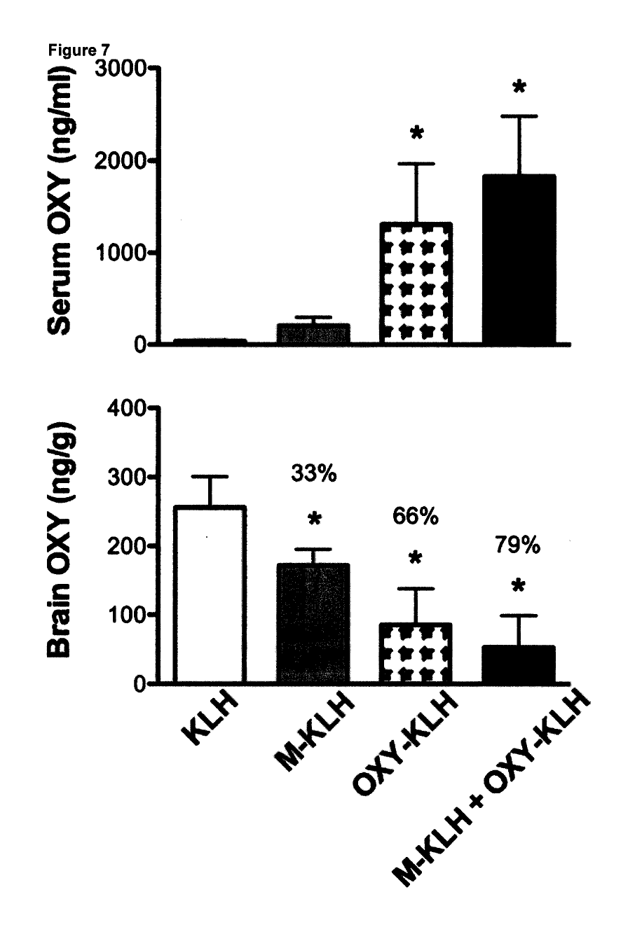

Monovalent M-KLH: Vaccination with M-KLH substantially increased the retention of 6-MAM and morphine in serum (FIG. 6). Distribution of 6-MAM to brain was reduced by 69% compared to controls. Morphine concentrations in brain in all groups were below assay sensitivity (<50 ng/ml). Serum anti-morphine antibody concentration was inversely correlated with the brain 6-MAM concentration. Vaccination with M-KLH also produced a small (33%) but significant decrease in distribution of oxycodone to brain (FIG. 7), consistent with ELISA titers showing some cross-reactivity of anti-morphine antibodies with the oxycodone immunogen.

The mean total number of IgG drug-binding sites in rats vaccinated with M-KLH was 0.98 μmol/kg, nearly 3-fold greater than the administered 6-MAM dose of 0.32 μmol/kg. The calculated saturation of serum anti-morphine IgG with its ligands (6-MAM+morphine) in the monovalent M-KLH group was 54±15% (mean±SD).

Monovalent OXY-KLH: Vaccination with OXY-KLH increased retention of oxycodone in serum and reduced oxycodone distribution to brain by 66%, FIG. 7). OXYM concentrations in all groups were too low to quantitate (<5 ng/ml). Vaccination with OXY-KLH produced a small increase in serum retention of morphine (FIG. 6) but had no significant effect on serum or brain 6-MAM concentrations.

Bivalent M-KLH and OXY-KLH: Vaccination with the bivalent vaccine preserved the effects of the monovalent vaccines, significantly increasing 6-MAM and oxycodone retention in serum and decreasing their distribution to brain (FIGS. 6 and 7). The effects of the bivalent vaccine were generally similar to those of the monovalent vaccines alone. The bivalent vaccine increased serum retention of 6-AM significantly more than the monovalent M-KLH vaccine. However the bivalent vaccine produced less retention of morphine in serum than the monovalent M-KLH vaccine, possibly due to increased binding of the precursor 6-MAM by the bivalent vaccine resulting in less conversion to morphine. Brain (p=0.12) or serum (p=0.06) oxycodone concentrations did not significantly differ between the bivalent and monovalent groups, although the difference in serum concentrations approached significance.

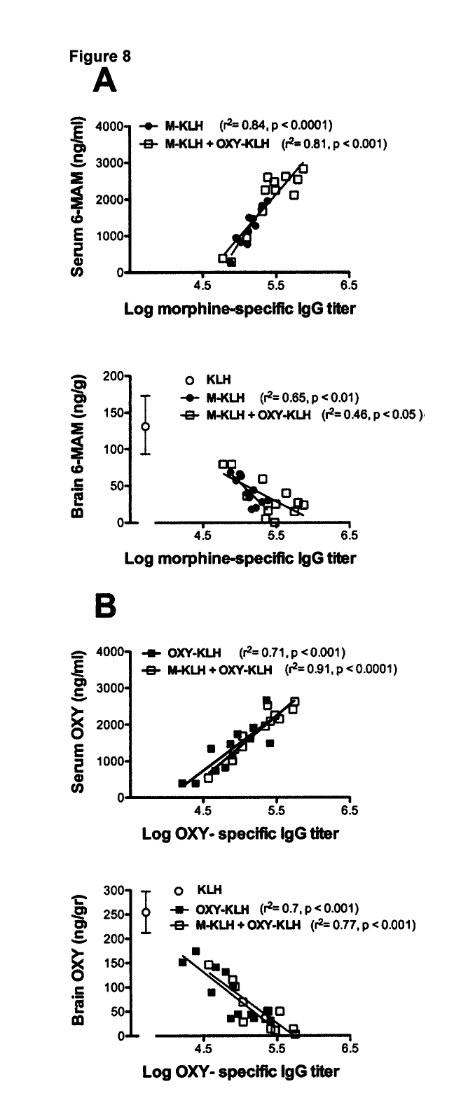

Vaccine effects on serum and brain 6-MAM or oxycodone concentrations were highly correlated with the corresponding serum antibody titers (FIG. 8). The relationships between drug-specific titers and drug concentrations regression slopes were not significantly different between the monovalent and bivalent groups (all p>0.2).

4. Discussion

Opioid vaccines are of interest as a potential adjunct to the treatment of opioid abuse or addiction but the wide variety of commonly abused opioids makes this challenging. Individual opioid vaccines reflect the high specificity of humoral antibody responses and each targets only a limited range of opioids. Vaccines based on morphine conjugates produce antibodies, which bind heroin and its active metabolites 6-MAM and morphine but have substantially lower affinity for oxycodone [14,19]. A vaccine based on an oxycodone conjugate produces antibodies, which bind oxycodone and its active metabolite oxymorphone but have substantially lower affinity for heroin and its metabolites [20]. In the current study combining M-KLH and OXY-KLH vaccines allowed their concurrent use while fully retaining their individual immunogenicity and effect on opioid pharmacokinetics. These observations provide proof of concept that a broad range of opioid coverage can be obtained through the use of multivalent vaccines.

The use of multivalent vaccines is well established for infectious diseases. Unrelated vaccines (e.g. DPT=diphtheria, pertussis, tetanus; MMR=measles, mumps, rubella) can be combined for convenience, or administered as separate injections at the same time, with little or no loss of efficacy. In principle the same should be true of conjugate vaccines constructed from small molecule haptens. This has been shown to be feasible for nicotine vaccines through the use of different linker positions to create immunologically distinct haptens [23]. Each of these haptens activated different populations of B cells to produce distinct populations of antibodies to nicotine. The current study extends this approach to opioids with the goal of targeting a wider range of opioids.

The efficacy of the M-KLH vaccine was studied using 6-MAM, which has a higher affinity for the mu opioid receptor than heroin and is present in brain at higher concentrations. 6-MAM is considered the principal mediator of the early reinforcing and rewarding effects of heroin [21]. A number of morphine-conjugate vaccines similar to M-KLH, which also utilize the C6 linker position, have been shown to block heroin self-administration in animals [14,15,18]. While the influence of M-KLH on heroin pharmacokinetics needs to be specifically studied, the high serum concentration of antibodies produced is comparable to the highest reported with other morphine-conjugate vaccines [15].

The OXY-KLH vaccine targeted oxycodone but the resulting antibodies also had a high affinity for oxymorphone. Oxymorphone is only a minor metabolite of oxycodone in humans but it is marketed separately as an analgesic medication. Tighter regulation of oxycodone prescribing and reformulation of oxycodone into tablets that are more difficult to dissolve for intravenous injection has led to increased abuse of alternative opioids including oxymorphone [6]. The potential use of the OXY-KLH vaccine to block oxymorphone distribution and effects is therefore of interest. Similarly, the relatively low IC50 values of both the M-KLH and OXY-KLH vaccines for hydrocodone suggest their efficacy might extend to this drug as well.

Antibodies generated by both vaccines showed some cross reactivity as measured by binding to immunogen conjugates on ELISA plates. Cross-reactivity in the setting of a bivalent vaccine is beneficial, as it allows both immunogen to contribute to binding either 6-MAM or oxycodone. Antibody titers to each immunogen in the bivalent group were however higher than could be accounted for by ELISA cross-reactivity alone. Since antibodies may bind differentially to the drug hapten alone compared to the corresponding hapten-linker immunogen [24], it is possible that there was additional cross reactivity of antibodies that was not detected by ELISA. The ELISA used immunogens with linkers attached, which may cover some epitopes that free drug in serum could nevertheless bind to. It is also possible that antibody affinity was enhanced in the bivalent group, but the generally similar IC₅₀ values for bivalent and monovalent vaccines suggest this was not the case. Consistent with enhanced titers in the bivalent group, 6-MAM retention in serum was greater and distribution to brain lower compared to the monovalent M-KLH group. Morphine retention in serum was less in the bivalent group, perhaps because increased binding of 6-MAM led to less conversion to morphine. Oxycodone concentrations showed a similar but non-significant trend toward greater efficacy in the bivalent group. Nevertheless, the most important result from the drug level measurements is confirmation that the two vaccines maintained their efficacy and did not interfere with each other when combined.

A limitation with available nicotine and cocaine vaccines has been the high variability in antibody titers and concentrations they produce in both animals and humans, with some subjects having levels too low to produce their desired effect [12,13,25-27]. The absence of such non-responders to M-KLH or OXY-KLH vaccines, and the very high serum antibody concentrations produced, suggests that their efficacy may be more uniform than that of nicotine or cocaine vaccines. Morphine-conjugate vaccines studied by others have also reported uniformly high antibody titers or concentrations and it may be that opioids in general are particularly effective haptens compared to nicotine or cocaine [15,19]. A cautionary note regarding the high serum antibody concentrations produced by opioid vaccines is that it is often more difficult to achieve very high serum antibody concentration in humans than it is in experimental animals. The reasons for this are not entirely clear. The current study used the i.p. route and Freund's adjuvant in rats, which are not appropriate for use in humans. However vaccination of mice by the s.c. route with alum adjuvant, which is acceptable in humans, generated anti-oxycodone antibody titers as high as vaccination i.p. with Freund's. Other heroin or morphine vaccine studies have also successfully used the s.c. route and alum adjuvant. Whatever the reason, it may be challenging to produce serum antibody concentrations in humans as high as those measured in the current study. Because antibody concentrations were uniformly high, our data do not directly comment on whether such high concentrations were necessary to achieve substantial efficacy. However magnitude of effect did correlate with antibody titer or concentration within the range of values measured, and this will be an important question to address.

A limitation of this study is that animals received only a single dose of opioid rather than repeated or chronic administration, and only one dose size. Because serum antibody concentrations were quite high, the calculated number of antibody drug-binding sites provided by M-KLH vaccination (0.98 μmol/kg) exceeded the dose of 6-MAM administered (0.32 μmol/kg). It may be more challenging to block the effects of higher or repeated 6-MAM doses. Also, effects of vaccination on opioid pharmacokinetics were evaluated but vaccine effects on opioid-induced behaviors were not. However the OXY-KLH vaccine, used alone, has already been shown to reduce oxycodone distribution to brain at a 5-fold higher i.v. oxycodone dose and to block hot plate analgesia induced by a 20-fold higher s.c. oxycodone dose [20]. In addition, several morphine conjugate vaccines have been shown to block heroin or morphine self-administration in rats, which involves repeated drug dosing [14,19]. These questions need to be addressed for the bivalent vaccine, but similar efficacy seems likely. The possible role of vaccines in addiction treatment is unclear because these vaccines are in early stages of development and the vaccines that have reached clinical trials to date have not had satisfactory immunogenicity [12]. It is unlikely that addiction vaccines will replace current therapies but they could serve as useful adjuncts. An opioid vaccine could be useful for opioid abusers who refuse agonist or antagonist treatment or who do not qualify for it because their use is occasional. Opioid vaccines may also be usable in combination with agonist therapy to provide efficacy during periods of agonist medication noncompliance or for those who persistently abuse opioids even while on agonist therapy. Opioid vaccines like M-KLH and OXY-KLH have been designed so that they target the desired opioids but not opioids, which may be needed for therapeutic use such as methadone, buprenorphine or naltrexone. The utility of this approach is not known, but highly immunogenic vaccines provide a tool for studying its potential. A bivalent vaccine which blocks a wide range of abusable opioids may have distinct advantages in areas of the world, like the USA, in which many different opioids are available and abused.

### Example 2 References

[1] National Survey on Drug Use and Health: National Findings. U.S. Department Of Health And Human Services Substance Abuse and Mental Health Services Administration Office of Applied Studies 2010; http://www.oas.samhsa.gov/nsduh/2k8nsduh/2k8Results.cfm.
[2] Maxwell J C. The prescription drug epidemic in the United States: a perfect storm. Drug and Alcohol Review 2011; 30(3):264-70.
[3] Compton W M, Volkow N D. Abuse of prescription drugs and the risk of addiction. Drug and Alcohol Dependence 2006; 83(Suppl. 1):S4-7.
[4] Compton W M, Volkow N D. Major increases in opioid analgesic abuse in the United States: concerns and strategies. Drug and Alcohol Dependence 2006; 81(2):103-7.
[5] 2008 Department of Defense Survey of Health Related Behaviors Among Active Duty Military Personnel. Department of Defense 2009; http://www.tricare. mil/2008HealthBehaviors.pdf.
[6] Oxymorphone Abuse: A growing Threat Nationwide. Department of Justice 2011; EWS Report 000011; http://www.justice.gov/ndic/pubs44/44817/sw0011p.pdf.
[7] Epidemic: Responding to America's Prescription Drug Abuse Crisis. Executive office of the president of the United States 2011; http://www.whitehouse.gov/sites/default/files/ondcp/policy-and-research/rx abuse plan.pdf.
[8] Butler S F, Black R A, Cassidy T A, Dailey T M, Budman S H. Abuse risks and routes of administration of different prescription opioid compounds and formulations. Harm Reduction Journal 2011; 8:29.
[9] Dodrill C L, Helmer D A, Kosten T R. Prescription pain medication dependence. American Journal of Psychiatry 2011; 168(5):466-71.
[10] Wu L T, Woody G E, Yang C, Blazer D G. How do prescription opioid users differ from users of heroin or other drugs in psychopathology: results from the National Epidemiologic Survey on Alcohol and Related Conditions. Journal o Addiction Medicine 2011; 5(1):28-35.
[11] Chi K R. Vaccines move forward against a range of addictions. Nature Medicine 2011; 17(2):146.
[12] Hatsukami D K, Jorenby D E, Gonzales D, Rigotti N A, Glover E D, Oncken C A, et al. Immunogenicity and smoking-cessation outcomes for a novel nicotine immunotherapeutic. Clinical Pharmacology and Therapeutics 2011; 89(3):392-9.
[13] Martell B A, Orson F M, Poling J, Mitchell E, Rossen R D, Gardner T, et al. Cocaine vaccine for the treatment of cocaine dependence in methadone maintained patients: a randomized, double-blind, placebo-controlled efficacy trial. Archives of General Psychiatry 2009; 66(10):1116-23.
[14] Anton B, Leff P. A novel bivalent morphine/heroin vaccine that prevents relapse to heroin addiction in rodents. Vaccine 2006; 24(16):3232-40.
[15] Anton B, Salazar A, Flores A, Matus M, Marin R, Hernandez J A, et al. Vaccines against morphine/heroin and its use as effective medication for preventing relapse to opioid addictive behaviors. Human vaccines 2009; 5(4): 214-29.
[16] Berkowitz B, Spector S. Evidence for active immunity to morphine in mice. Science 1972; 178(67):1290-2.
[17] Berkowitz B A, Ceretta K V, Spector S. Influence of active and passive immunity on the disposition of dihydromorphine-H3. Life Sciences 1974; 15(5):1017-28.
[18] Bonese K F, Wainer B H, Fitch F W, Rothberg R M, Schuster C R. Changes in heroin self-administration by a rhesus monkey after morphine immunisation. Nature 1974; 252(5485):708-10.
[19] Stowe G N, Vendruscolo L F, Edwards S, Schlosburg J E, Misra K K, Schulteis G, et al. A vaccine strategy that induces protective immunity against heroin. Journal of Medicinal Chemistry 2011; 54(14):5195-204.
[20] Pravetoni M, Le Naour M, Harmon T, Tucker A, Portoghese P S, Pentel P R. An oxycodone conjugate vaccine elicits oxycodone-specific antibodies that reduce oxycodone distribution to brain and hot-plate analgesia. Journal of Pharmacology and Experimental Therapeutics 2012.
[21] Andersen J M, Ripel A, Boix F, Normann P T, Morland J. Increased locomotor activity induced by heroin in mice: pharmacokinetic demonstration of heroin acting as a prodrug for the mediator 6-monoacetylmorphine in vivo. Journal of Pharmacology and Experimental Therapeutics 2009; 331(1):153-61.
[22] Bazin-Redureau M I, Renard C B, Scherrmann J M. Pharmacokinetics of heterologous and homologous immunoglobulin G: F(ab′)2 and Fab after intravenous administration in the rat. Journal of Pharmacy and Pharmacology 1997; 49(3):277-81.
[23] Pravetoni M, Keyler D E, Pidaparthi R R, Carroll F I, Runyon S P, Murtaugh M P, et al. Structurally distinct nicotine immunogens elicit antibodies with nonoverlapping specificities. Biochemical Pharmacology 2011.
[24] Tars K, Kotelovica S, Lipowsky G, Bauer M, Beerli R R, Bachmann M F, et al. Different binding modes of free and carrier-protein-coupled nicotine in a human monoclonal antibody. Journal of Molecular Biology 2012; 415(1):118-27.
[25] Haney M, Gunderson E W, Jiang H, Collins E D, Foltin R W. Cocaine-specific antibodies blunt the subjective effects of smoked cocaine in humans. Biological Psychiatry 2010; 67(1):59-65.
[26] Kantak K M, Collins S L, Bond J, Fox B S. Time course of changes in cocaine self-administration behavior in rats during immunization with the cocaine vaccine IPC-1010. Psychopharmacology 2001; 153(3):334-40.
[27] Pravetoni M, Keyler D E, Raleigh M D, Harris A C, Lesage M G, Mattson C K, et al. Vaccination against nicotine alters the distribution of nicotine delivered via cigarette smoke inhalation to rats. Biochemical Pharmacology 2011; 81(9):1164-70.

### Example 3

### Reduced Antinociception of Opioid in Rats and Mice by Vaccines Containing Oxycodone and Hydrocodone Haptens

Vaccination against prescription opioids may provide an alternative to pharmacotherapy. An oxycodone (OXY) hapten containing a tetraglycine linker at the C6 position conjugated to keyhole limpet hemocyanin (6OXY(Gly)₄-KLH) has shown early proof-of-efficacy in rodents as a candidate vaccine for the treatment of oxycodone abuse. In this study, haptens based on OXY and hydrocodone modified at the C6 position and hydrocodone modified at the C8 position were conjugated to KLH to generate vaccines that would recognize both OXY and hydrocodone. Vaccination with 6OXY(Gly)₄-KLH increased drug binding in serum, reduced drug distribution to brain and blunted analgesia for both OXY and hydrocodone. An analogous C6-linked hydrocodone vaccine blocked hydrocodone effects but less so than 6OXY(Gly)₄-KLH. C8-linked hydrocodone immunogens had only limited efficacy. Amide conjugation showed higher haptenation ratios and greater efficacy than thioether conjugation. The 6OXY(Gly)₄-KLH vaccine may be of potential use for treatment of prescription opioid abuse.

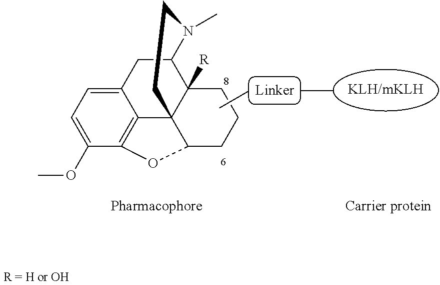

Introduction

Prescription opioid abuse is increasing and is now more common than heroin abuse in the USA¹. Oxycodone, oxymorphone and hydrocodone are the most commonly abused prescription opioids^{1a, b, 2}. Treatment of prescription opioid abuse is complex because addicts may switch or transition between different prescription opioids to overcome lack of availability³. The prevalence of prescription opioid abuse highlights the need for effective therapies.

Vaccines to treat addiction are a promising alternative to current pharmacotherapies. Addiction vaccines act by stimulating production of drug-specific antibodies that bind and retain the target drug in serum. Vaccination reduces the distribution of drug to the brain and its ability to have central effects, thus attenuating behavioral effects. Proof-of-principle of clinical efficacy has been shown with nicotine and cocaine vaccines⁴.

Several groups have developed immunogens based upon the morphine structure to block the effects of heroin and its active metabolites 6-monoacetylmorphine and morphine⁵. Heroin/morphine vaccine efficacy has been established in animal models but has not been evaluated in humans. Fewer studies have focused on development of immunogens to target prescription opioids such as oxycodone, oxymorphone or hydrocodone^{5d, 6}.

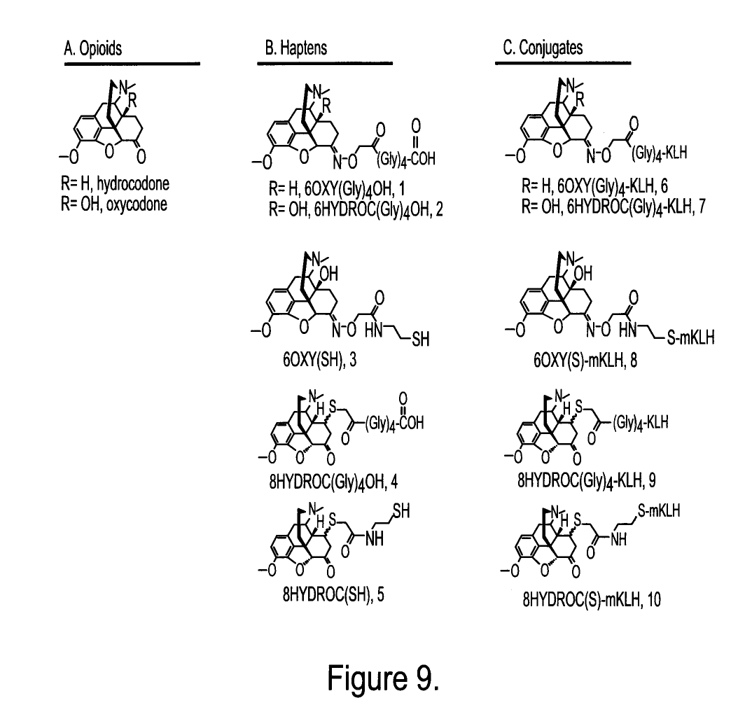

Evaluation of a recently described oxycodone vaccine showed that modifying oxycodone at the C6 position and conjugating this hapten to a carrier protein through a tetraglycine linker provided an immunogen that generates a strong immune response in mice and rats^{6b 5d}. The tetraglycine linker was more effective than a shorter succinic acid based linker^{6b}. Vaccination with 6OXY(Gly)₄-KLH (6, FIG. 9) elicited antibodies that recognize oxycodone and its active metabolite oxymorphone, and reduced oxycodone distribution to brain and blocked its analgesia^{6b}.

In the current study, we adopted an analogous approach and used the C6 position and the tetraglycine linker to generate a candidate hydrocodone vaccine (7, FIG. 9), and used the C8 position to generate the hapten 8HYDROC(Gly)₄OH (4). At both positions, the thioether-maleimide conjugation method was compared to amide linkage for coupling haptens 3 and 5 to maleimide-activated KLH (mKLH). The resulting immunogens 8 and 10 were compared to the previously characterized 6OXY(Gly)₄-KLH (6). The effect of vaccination on the distribution of oxycodone and hydrocodone to brain was determined in rats. Vaccine efficacy in blunting oxycodone and hydrocodone behavioral effects was determined in a test of thermal nociception in mice and rats. Results show that the previously described oxycodone vaccine 6 had efficacy against hydrocodone as well as oxycodone, and that haptens 1 and 3 were more effective than the hydrocodone based haptens 2, 4 and 5.

Results

Hapten Design and Synthesis

Critical parameters for the generation of hapten-protein conjugate vaccines are the derivatization site on the hapten to which the linker is attached, linker length, the conjugation method used, and the immunogenicity of the carrier protein. In prior studies, a 12 atom tetraglycine linker was found superior to a shorter linker for an oxycodone hapten^{6b}. For these reasons, this linker was used to generate tetraglycine-containing hydrocodone haptens. In general the immunogenicity of hapten-protein conjugate vaccines is improved with a higher ratio of haptenation, so this study investigated if thiol-based maleimide conjugation would improve haptenation ratios compared to the carbodiimide method of conjugation. The synthesis of haptens 2 and 3 is shown in Scheme 2.

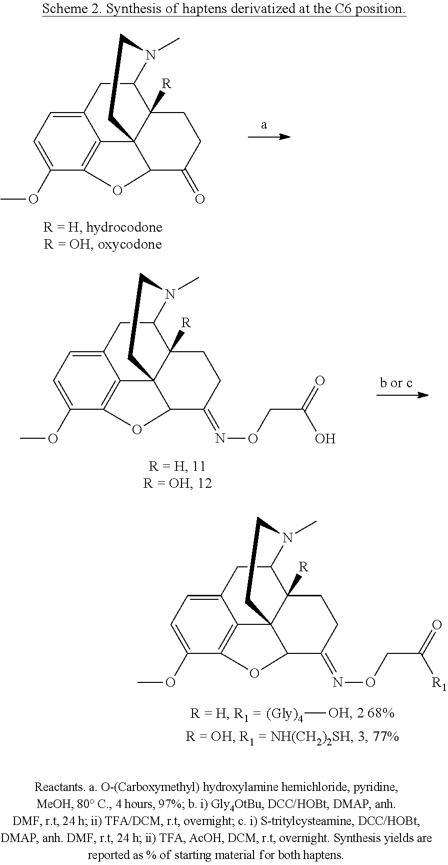

To generate a hydrocodone vaccine, a close analogue of our lead oxycodone hapten was synthesized using hydrocodone as starting material. Conditions similar to those previously described^{6b} led to intermediate 11⁷. Carboxylic acids 11 and 12 were then coupled to linkers Gly₄OtBu and S-tritylcysteamine⁸ using the dicyclohexylcarbodiimide/hydroxybenzotriazole (DCC/HOBt) procedure. Hydrolysis of the tertbutyl ester protecting group with trifluoroacetic acid (TFA) in dichloromethylene (DCM) produced hapten 2 in good yield (68% overall). The precursor of the sulfhydryl-containing hapten 12 was detritylated in situ by treatment with acetic acid (AcOH) and trifluoroacetic acid in dichloromethane to give the free thiol 3 in a similar yield (77%). In order to investigate the role of the 6-keto group on the opioid scaffold, these linkers were also attached at the C8 position. Thus, the Michael addition of thioglycolic acid to codeinone⁹ (Scheme 3) allowed modification at the C8 position with retention of the 6-keto-group of the corresponding 8-substituted dihydrocodeinone (4 and 5).

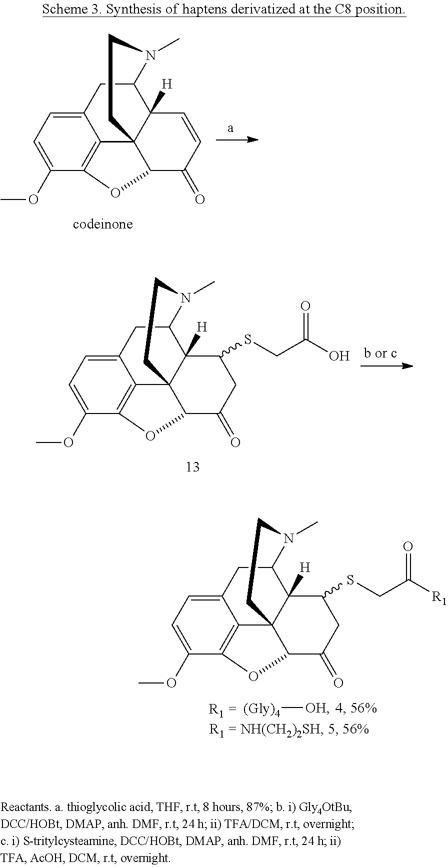

Under these conditions, the reaction afforded a mixture of 8α- and 8β-isomers (ratio 25/75) as determined by NMR. Assignment of the ¹H NMR of the mixture of α- and β-epimers was made possible as H1 and H5 had a slightly different chemical shift between the 2 isomers. Integration of the two peaks showed a ratio of 75/25 in favor of the 8β-isomer as reported in similar studies¹⁰. The identification of the β-adduct as the major isomer is consistent with the less hindered 8β position of the morphinan scaffold. As the isomers could not be separated by conventional chromatographic methods, the isomeric mixture was used for conjugation to proteins.

Conjugation of Haptens to Carrier Proteins

Haptenation ratios for the KLH conjugates could not be measured due to the large size of KLH, so conditions were standardized using BSA as a model carrier protein. Haptenation ratios for 6OXY(Gly)₄OH (1) and 8HYDROC(Gly)₄OH (4) to BSA or OVA were comparable and ranged from 17 to 21 moles of hapten per mole of carrier protein as determined by MALDI-TOF. The 6HYDROC(Gly)₄OH (2) exhibited molar haptenation ratios of 12 to 14 bound to BSA.

Activation of BSA resulted in 15 to 17 maleimide per mole of BSA, as determined by MALDI-TOF, similar to activated proteins from commercial sources and in the same range as previous reports by our and other groups¹¹. Haptenation ratios for 6OXY(S)-mBSA were ˜5-10 and similar to previously described haptens conjugated through maleimide^{11a}, although higher haptenation ratios have been reported with a morphine hapten^{5e}. The haptenation ratio was lower for hapten 5 conjugated to mBSA. To minimize concerns of maleimide activation efficacy, hapten 3 conjugated to commercially available mKLH was compared to vaccine 8 containing the in-house mKLH by evaluation of the effect of vaccination on analgesia in mice, but was not found to be more effective.

### Biological Studies

### Experiment I

### Effect of Vaccination on Oxycodone Distribution after Intravenous Drug Dosing in Rats

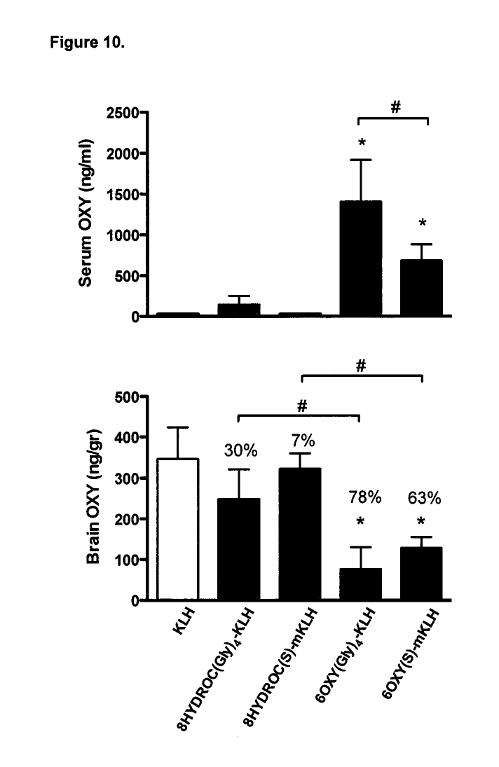

In rats immunized with either 6OXY(Gly)₄-KLH (6) or 6OXY(S)-mKLH (8) immunogens, the retention of oxycodone in serum was increased compared to unconjugated KLH control vaccine at 5 minutes after the oxycodone dose (p<0.05, FIG. 10, upper panel). The 6OXY(Gly)₄-KLH (6) vaccine was more effective than 6OXY(S)-mKLH (8) (p<0.05, see FIG. 10, upper panel). The 8HYDROC(Gly)₄-KLH (9) and 8HYDROC(S)-mKLH (10) vaccines did not increase the serum oxycodone concentration. Both 6OXY(Gly)₄-KLH (6) and 6OXY(S)-mKLH (8) immunogens significantly reduced the distribution of oxycodone to brain (p<0.05, FIG. 10, lower panel). The effect of vaccination on oxycodone distribution was confirmed by showing dramatically increased protein binding of oxycodone in serum from 14±3 to 100±2% (Table 5); the unbound drug concentrations were not different across groups, but there was considerable variability in serum concentrations and a trend toward lower unbound concentration in the 6OXY(Gly)₄-KLH (6) group.

TABLE 5
Serum protein binding of oxycodone or hydrocodone in rats.

| | | | |
|---|---|---|---|
| | Total drug | Unbound drug | Bound Drug |
| Vaccine | ng/ml | ng/ml | % |

5 min after 0.1 mg/kg oxycodone i.v.

| | | | |
|---|---|---|---|
| KLH | 30 ± 2 | 20 ± 4 | 14 ± 10 |
| 6OXY(Gly)₄-KLH (6) | 1400 ± 510***^{, ###} | 20 ± 40 | 100 ± 2***^{, #} |
| 8HYDROC(Gly)₄-KLH (9) | 140 ± 110 | 30 ± 14 | 80 ± 17*** |

30 min after 2.25 mg/kg oxycodone s.c.

| | | | |
|---|---|---|---|
| KLH | 330 ± 60 | 300 ± 40 | 10 ± 5 |
| 6OXY(Gly)₄-KLH (6) | 8400 ± 8600 | 280 ± 580 | 100 ± 3***^{, ###} |
| 8HYDROC(Gly)₄-KLH (9) | 690 ± 210 | 250 ± 80 | 60 ± 14*** |

30 min after 6.75 mg/kg hydrocodone s.c.

| | | | |
|---|---|---|---|
| KLH | 660 ± 170 | 440 ± 240 | 30 ± 30 |
| 6OXY(Gly)₄-KLH (6) | 1900 ± 410***^{, ###} | 210 ± 120 | 90 ± 10***^{, ###} |
| 6HYDROC(Gly)₄-KLH (7) | 610 ± 280 | 360 ± 140 | 35 ± 20 |

Group sizes were n = 5 for oxycodone and n = 7-12 for hydrocodone; data are expressed as mean ± SD;
* p < 0.05,
** 0.01 and
***0.001 compared to KLH controls;
^{#} p < 0.05,
^{###} p < 0.001 compared to other vaccine.

The effect of vaccination on oxycodone distribution was further confirmed by the higher oxycodone-specific serum antibody titers generated by 6 compared to 9 (Table 6). Oxycodone-specific serum antibody titer generated from the 8 and 10 immunogens are not reported because of apparent interference in the ELISA with use of the SH linkage.

TABLE 6
Serum antibody titers (×10³)^{#}.

Vaccine

| Coating | 6OXY(Gly)₄- | 8HYDROC(Gly)₄- |
|---|---|---|
| immunogen | KLH (6) | KLH (9) |
| 6OXY(Gly)₄-OVA | 490 ± 397 | 66 ± 85 |
| 8HYDROC(Gly)₄-OVA | 71 ± 2 | 150 ± 190 |
| 6OXY(Gly)₄-BSA | 190 ± 71 | 77 ± 92 |
| 8HYDROC(Gly)₄-BSA | 3 ± 1 | 76 ± 48 |

^{#}Data are expressed as mean ± SD.
Chicken Ovalbumin (OVA)

### Experiment II

### Effect of Vaccination on Oxycodone and Hydrocodone Distribution and Analgesia after Subcutaneous Drug Dosing in Rats

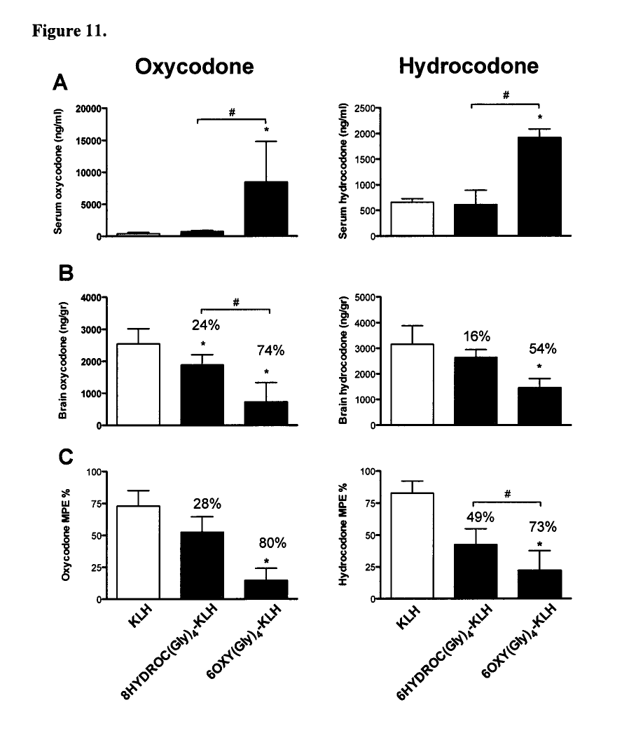

6OXY(Gly)₄-KLH (6) was more efficient than 8HYDROC(Gly)₄-KLH (9) in blocking oxycodone analgesia and distribution to brain (FIG. 11), and also markedly increased oxycodone protein binding in serum from 10±5 to 100±3% (Table 5). In these rats, no titers are available since trunk blood was collected by euthanasia after drug administration; the presence of oxycodone interferes with measurement of serum antibody titers by ELISA (data not shown).

The 6OXY(Gly)₄-KLH (6) immunogen elicited serum antibody titers of 142,000±103,000 (mean±SD) which were significantly higher than those elicited by 6HYDROC(Gly)₄-KLH (7) (50,000±48,000, p<0.05). In experiment II, immunogen 6 elicited lower titers than reported in experiment I (Table 6), which may be attributable to the lower immunogen dose used in this experiment. The 6OXY(Gly)₄-KLH (6) immunogen generated a stronger blockage of hydrocodone analgesia than 6HYDROC(Gly)₄-KLH (7) (p<0.05; FIG. 11, panel C). This finding is consistent with the greater effect of 6 on hydrocodone distribution and protein binding (FIG. 11, panel B and Table 5). Rats immunized with 6OXY(Gly)₄-KLH (6) immunogen showed a stronger reduction in brain oxycodone (74%, p<0.05 compared to KLH control, FIG. 11) than brain hydrocodone (54%, p<0.05 compared to KLH control, FIG. 11). 6OXY(Gly)₄-KLH (6) comparably attenuated both oxycodone and hydrocodone analgesia in rats, but fentanyl analgesia was preserved (data not shown) at 82±18 (mean±SEM) MPE %.

### Experiment III

### Effect of Vaccination on Oxycodone Analgesia after Subcutaneous Drug Dosing in Mice

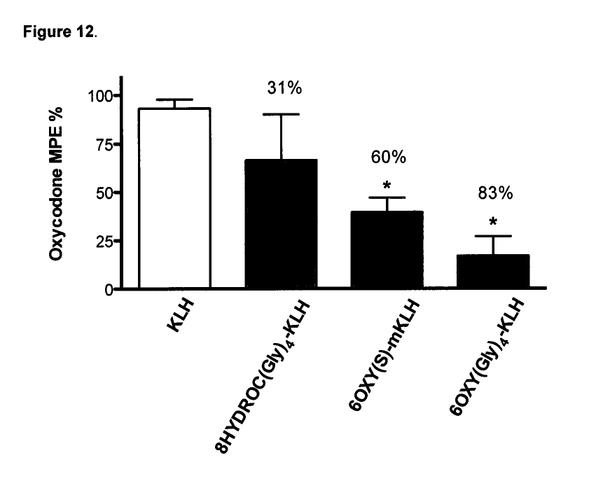

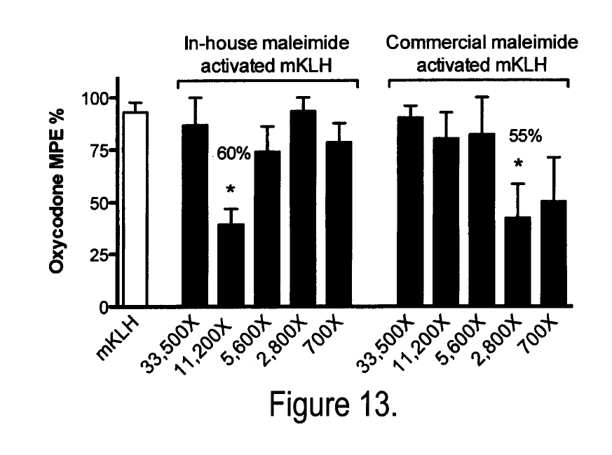

To investigate generality across species and using a different adjuvant and route of administration (alum, s.c.), the effect of vaccination with the 6OXY(Gly)₄-KLH (6), 6OXY(S)-mKLH (8) or 8HYDROC(Gly)₄-KLH (9) vaccines was compared using hot plate oxycodone analgesia in BALC/c mice. Similar to rats, mice immunized with 6 and 8 showed a significant decrease in oxycodone behavioral effects compared to KLH controls (p<0.05, FIG. 12). 6OXY(S)-mKLH (8) conjugation reaction conditions were optimized through changes of hapten to protein ratios and evaluated for their ability to block oxycodone analgesia in mice (FIG. 13). Conjugation conditions used a range of hapten to carrier protein ratios previously shown to be effective with a nicotine hapten conjugated through maleimide chemistry^{11b}.

Discussion

The main finding of this study was that haptens based on modification of oxycodone at the C6 position produced more effective immunogens than hydrocodone haptens modified at the C6 or C8 positions for eliciting antibodies against both oxycodone and hydrocodone, and blocking their distribution to brain or their behavioral effects. Use of a (Gly)₄OH linker and amide linkage to KLH resulted in a higher protein haptenation ratios than thioether linkage to mKLH, and greater efficacy. These data suggest that the previously characterized 6OXY(Gly)₄-KLH (6) vaccine may be useful in treating both oxycodone and hydrocodone abuse.

Linker position and composition of hapten-protein conjugate vaccines are important determinants of their immunogenicity but the number of haptens attached to each molecule of protein also profoundly affects immunogenicity. Haptenation ratios are not always measured when investigating or comparing conjugate vaccines, so it can be difficult to know whether an immunogen with greater immunogenicity is intrinsically more stimulating to the immune system, or simply more efficiently linked to its carrier protein so that it provides a higher density of hapten epitopes. KLH is a commonly used carrier protein for small molecule haptens because of its efficacy and acceptability for human use. KLH haptenation ratios cannot be measured by mass spectrometry because of its large size, so conjugation reactions in the current study were first carried out using BSA as a carrier protein that allows such measurement as well as optimization of conjugation conditions. We have found previously that conjugation procedures that increase the haptenation and immunogenicity of nicotine-BSA conjugates also increase the immunogenicity of the corresponding nicotine-KLH conjugates^{11b}. Haptenation ratios to BSA were therefore used as a surrogate for a direct measure of KLH haptenation.

Because the previously studied 6OXY(Gly)₄-KLH (6) was highly effective in blocking oxycodone effects in rats, the analogous 6HYDROC(Gly)₄-KLH (7) was anticipated to be similarly immunogenic against hydrocodone and effective in blocking its effects. The 6HYDROC(Gly)₄-KLH (7) immunogen did have activity in blocking hydrocodone distribution and antinociception but, surprisingly, less so than 6OXY(Gly)₄-KLH (6). The cross-reactivity of antibodies generated by 6OXY(Gly)₄-KLH (6) suggests that the presence of substitution at the 14-position, the only structural difference between oxycodone and hydrocodone, is not recognized by the antibodies generated by this immunogen. The lesser immunogenicity of the hydrocodone based immunogen may have been due to the possibility that 6HYDROC(Gly)₄OH (2) is less efficient for haptenation of KLH than 6OXY(Gly)₄OH (1). However, this seems less likely given that the haptenation ratio was only 10% lower.

The 8-substituted 8HYDROC(Gly)₄-KLH (9) had only limited efficacy in blocking hydrocodone analgesia despite having a protein haptenation ratio equal to that of the most effective immunogen, 6OXY(Gly)₄-KLH (6). Since this immunogen was derived from an unresolvable mixture of 8α- and 8β-epimers, respectively 25/75, the density of the active isomer on KLH may have been lower. However it has been shown that specific antibodies can be selectively generated for each isomer of a racemic hapten mixture¹². The 8HYDROC(Gly)₄-KLH (9) immunogen was also less effective than both C6-linked OXY haptens even though 6OXY(S)-mKLH (8) had a substantially lower haptenation ratio than 8HYDROC(Gly)₄-KLH (9). This suggests that the 6OXY hapten structure is intrinsically more immunogenic than the 8HYDROC hapten structure and that the absence of substitution at C8 is important for preserving this immunogenicity.

Maleimide activation of carrier proteins resulted in a number of maleimide groups attached to mBSA similarly to activated proteins from commercial sources and in the same range as previous reports by our and other groups^{11a, 13}. The 6OXY(S)-mBSA conjugate showed haptenization ration similar to previously described haptens conjugated through maleimide^{11a}, although higher haptenization ratios have been reported with a maleimide-containing morphine conjugate^{5e}. Despite a relatively low haptenation ratio, the immunogen 8 elicited an efficient immune response that blocked the distribution and the behavioral effects of oxycodone. These data suggest that if the haptentation were further improved, 6OXY(S)-mKLH (8) could be an alternative to 6OXY(Gly)₄-KLH (6).

Linker length was not a focus of this study. The 12 atom (Gly)₄OH linker was used because it has been found to be suitable and superior to shorter linkers containing 0-2 glycines, for an analogous morphine vaccine. The linker used for the SH immunogens was shorter than (Gly)₄OH but its effective length is similar given that, like the carbodiimide conjugates it attaches to mKLH through the ε-amino group of lysine residues. In a previous study 6OXY(Gly)₄-KLH (6) was compared with an oxycodone immunogen containing a shorter hemisuccinate-like linker at the C6 position, and the tetraglycine linker was more effective in evoking serum antibody titers^{6b}.

Because the purpose of this study was to identify an effective hydrocodone immunogen, not all immunogens were compared in each assay. However all immunogens were compared to the lead compound 6OXY(Gly)₄-KLH (6) and by all measures this immunogen was the most effective. Prescription opioid abuse is a challenging treatment target because abusers may use different prescription opioids at different times. A vaccine that blocks both oxycodone and hydrocodone, two of the most commonly abused prescription opioids, might be of value. These data suggest that 6OXY(Gly)₄-KLH (6) is a reasonable candidate for generating polyspecific antibodies against both drugs, and for further investigation of the merit of this therapeutic approach.

The structure of fentanyl differs substantially from that of oxycodone and hydrocodone. As expected, rats vaccinated with the 6OXY(Gly)₄-KLH (6) vaccine did not show a significant reduction in fentanyl analgesia, indicating that the efficacy of this immunogen does not extend to all opioids. This may be therapeutically useful because it would allow individuals vaccinated with this immunogen to still obtain full effect from fentanyl or other opioids if required for their medical needs. On the other hand, if fentanyl abuse should emerge as a common problem, it may be necessary to combine 6OXY(Gly)₄-KLH (6) with an immunogen specifically targeting this drug.

Conclusions

A previously described oxycodone-based immunogen, 6OXY(Gly)₄-KLH, was found to generate polyspecific antibodies with activity against hydrocodone as well as oxycodone, and was more effective in doing so than hydrocodone based immunogens. The greater efficacy of this immunogen was likely due to both its intrinsic immunogenicity and a high protein haptenation ratio. The ability of 6OXY(Gly)₄-KLH to attenuate the distribution of both oxycodone and hydrocodone to brain, as well as their analgesic activity, identifies it as a useful tool for studying the potential use of vaccination to treat prescription opioid abuse.

### Experimental Section

Drugs and Reagents.

All opioids were obtained through the NIDA Drug Supply Program and Sigma (St. Louis, Mo.). All drug doses and concentrations are expressed as the weight of the base.

Synthesis of Oxycodone and Hydrocodone Haptens.

General Information.

NMR spectra were recorded on a Bruker Avance 400 MHz spectrometer (Bruker Daltonics, Billerica, Mass.). Chemical shifts are in parts per million (ppm). The assignments were made using one-dimensional ¹H and ¹³C spectra. ESI mass spectra were recorded on a BrukerBioTof II system. Preparative High-Performance Liquid Chromatography (HPLC) was performed using a VERSAPrep system with a Haisil column (100 C-18, 5 mm, 10 mm×250 mm). A gradient starting from 90% CH₃CN/10% H₂O/0.1% trifluoroacetic acid (TFA) reaching 70% CH₃CN/30% H₂O/0.1% TFA was used. Flash chromatography on silica gel were performed on EM Science silica gel 60 (230-400 mesh). A gradient starting from 2% methanol/97% dichloromethane (DCM)/1% NH₄OH reaching 10% methanol/89% DCM/1% NH₄OH was used. Purity (%) was determined by reverse phase HPLC, using UV detection (254 nm), and all compounds showed purity greater than 95%. Melting points were determined on a Thomas-Hoover melting point apparatus and are uncorrected. All commercial reagents and solvents were used without further purification.

6-Carboxymethyloxime Hydrocodone (11)

To a solution of hydrocodone base (300 mg, 1 mmol) in methanol (5 ml) were added pyridine (196 μl, 2 mmol) and carboxymethoxylamine hemichloride (262 mg, 1.2 mmol). The reaction was stirred at 80° C. for 4 hours. After the solvent was removed in vacuo, the crude residue was taken up and triturated in an acetone/diethyl ether mixture to afford 2 (97%) as a white solid. ¹H NMR (DMSO): 1.13 (m, 1H); 1.85-1.88 (m, 2H); 2.14 (m, 1H); 2.41-2.49 (m, 2H); 2.55 (s, 3H); 2.99-3.01 (m, 2H); 3.22-3.25 (m, 2H); 3.87 (s, 3H, OCH₃); 4.47 (s, 2H); 4.94 (s, 1H); 6.64 (d, 1H, J_{H1-H2}=8.1 Hz); 6.72 (d, 1H, J_{H2-H1}=8.1 Hz); ESI-TOF MS calculated for C₂₀H₂₄N₂O₃, m/z 372.415. found 373.367 (MH)⁺.

8-Thioacetic Acid Hydrocodone (13)

A solution of codeinone (⁹ 250 mg, 8.4 mmol) and thioglycolic acid (65 mL, 0.92 mmol) in anhydrous tetrahydrofurane (THF, 5 ml) was stirred at room temperature for 20 hours. A solid began to form almost immediately. The suspension was filtered, the resulting white solid washed with water and diethyl ether and dried to afford 13 (87%) as a 25/75 mixture of isomers. ¹H NMR (DMSO) β (major isomer): 1.52 (m, 1H); 2.05 (m, 2H); 2.37 (s, 3H, NCH₃), 2.41-2.59 (m, 5H); 2.77 (m, 1H); 2.94 (m, 2H); 3.30 (dd, 2H, J=6.4 Hz, J=15.4 Hz); 3.77 (s, 3H, OCH₃); 4.93 (s, 1H); 6.65 (d, 1H, J_{H1-H2}=8.1 Hz); 6.73 (d, 1H, J_{H2-H1}=8.1 Hz); ESI-TOF MS calculated for C₂₀H₂₃NO₅S, m/z 389.461. found 390.258 (MH)⁺.

General Procedure for Coupling and Deprotection of Haptens 2-5.

To a solution of carboxylic acid (1 mol equivalent) in N,N-dimethylformamide (DMF), amine (1.2 mol equivalent), dicyclohexylcarbodiimide (DCC, 2.0 mol equivalent), hydroxybenzotriazole (HOBt, 2 mol equivalent), and catalytic N,N-dimethylaminopyridine (DMAP, 0.1 mol equivalent) were sequentially added. After stirring at room temperature for 24 hrs, the reaction was evaporated, and the residue was dissolved in DCM and then filtered to remove dicyclohexylurea. The resulting solution was treated with TFA (10 mol equivalent) and glacial acetic acid (AcOH.10 mol equivalent) when a trityl protecting group was used. After stirring at room temperature for 16 hours, the solvent was removed by evaporation and the residue was purified by column chromatography on silica gel or on a reverse-phase HPLC column.

6HYDROC(Gly)₄OH (2)

Reacting amine GlyOtBu (180 mg, 0.6 mmol), acid 11 (185 mg, 0.5 mmol); DCC (205 mg, 1.0 mmol), HOBt (134 mg, 1.0 mmol) and DMAP (6 mg, 0.05 mmol) followed by treatment with TFA as reported above gave the target compound 2 which was purified by reverse-phase HPLC; product retention time: 9 min; white solid; yield 68%. ¹H NMR (CD₃OD) δ: 1.28 (m, 1H); 1.61-1.67 (m, 2H); 2.34-3.14 (m, 14H with s, 3H, NCH₃ at 2.67); 3.71 (m, 1H); 3.86 (s, 3H, OCH₃); 4.50 (s, 2H); 5.00 (s, 1H); 6.28 (t, 1H, NH, J=8. Hz); 6.73 (d, 1H, J_{H1-H2}=8.1 Hz); 6.82 (d, 1H, J_{H2-H1}=8.1 Hz); ¹³C NMR (DMSO) 17.82; 21.34; 28.45; 28.51; 31.64; 39.78; 41.34; 42.13; 42.33; 42.56; 42.63; 44.65; 49.37; 59.72; 70.20; 85.74; 116.74; 119.54; 126.70; 136.57; 142.40; 144.46; 156.68; 168.23; 171.0; 171.30; 171.32; 174.61; mp 176° C. (decomposition); Anal. Calcd. for C₂₈H₃₆N₆O₉: C, 55.99; H, 6.04; N, 13.99. Found: C, 55.83; H, 5.97; N, 13.87. ESI-TOF MS calculated for C₂₈H₃₆N₆O₉, m/z 600.620. found 601.548 (MH)⁺.

6OXY(SH) (3)

Reacting S-tritylcysteamine (121 mg, 0.38 mmol), acid 12 (122 mg, 0.31 mmol); DCC (128 mg, 0.62 mmol), HOBt (84 mg, 0.62 mmol) and DMAP (4 mg, 0.03 mmol) followed by treatment with TFA and AcOH as reported above gave the target compound 3 which was purified by flash chromatography on silica gel; white solid; yield 77%. NMR (DMSO) δ: 1.28 (m, 1H); 1.61-1.67 (m, 2H); 2.34-3.14 (m, 14H with s, 3H, NCH₃ at 2.67); 3.71 (m, 1H); 3.86 (s, 3H); 4.51 (s, 2H); 4.99 (s, 1H); 6.73 (d, 1H, J_{H1-H2}=8.1 Hz); 6.82 (d, 1H, J_{H2-H1}=8.1 Hz); ¹³C NMR (DMSO) 17.96; 23.25; 26.84; 27.34. 28.17; 28.42; 41.13; 45.26; 46.28; 56.32; 65.14; 69.47; 70.34; 85.27; 115.30; 119.68; 123.56; 128.84; 142.26; 144.46; 155.83; 170.66; mp 184° C.; ESI-TOF MS calculated for C₂₂H₂₉N₃O₅S, m/z 447.548. found 448.370 (MH)⁺.

8HYDROC(Gly)₄OH (4)

Reacting amine GlyOtBu (112 mg, 0.37 mmol), acid 13 (120 mg, 0.31 mmol); DCC (128 mg, 0.62 mmol), HOBt (84 mg, 0.62 mmol) and DMAP (4 mg, 0.03 mmol) followed by treatment with TFA as reported above gave the target compound 4 which was purified by reverse-phase HPLC; Product retention time: 12 min; slightly brownish solid; yield 56%. ¹H NMR (DMSO) (major isomer): 1.53 (m, 1H); 2.05 (m, 2H); 2.32 (s, 3H, NCH₃), 2.39-2.47 (m, 5H); 2.75 (m, 1H); 2.91 (m, 2H); 3.31-3.35 (m, 10H); 3.78 (s, 3H, OCH₃); 4.95 (s, 1H); 6.62 (d, 1H, J_{H1-H2}=8.1 Hz); 6.72 (d, 1H, J_{H2-H1}=8.1 Hz); ESI-TOF MS calculated for C₂₈H₃₅N₅O₉S, m/z 617.671. found 618.723 (MH)⁺.

8HYDROC(SH) (5)

Reacting S-tritylcysteamine (83 mg, 0.34 mmol), acid 13 (110 mg, 0.28 mmol); DCC (115 mg, 0.56 mmol), HOBt (76 mg, 0.56 mmol) and DMAP (4 mg, 0.03 mmol) followed by treatment with TFA and AcOH as reported above gave the target compound 5 which was purified by reverse-phase HPLC; Product retention time: 10 min; slightly brownish solid; yield 56%. ¹H NMR (CD₃OD) (major isomer): 1.53 (m, 1H); 2.07 (m, 2H); 2.35 (s, 3H, NCH₃), 2.40-2.62 (m, 7H); 2.72-2.81 (m, 3H); 2.95 (m, 2H); 3.78 (s, 3H, OCH₃); 4.94 (s, 1H); 6.67 (d, 1H, J_{H1-H2}=8.1 Hz); 6.71 (d, 1H, J_{H1-H2}=8.1 Hz); ESI-TOF MS calculated for C₂₂H₂₈N₂O₄S₂, m/z 448.599. found 449.641 (MH)⁺.

Conjugation of 6, 7 and 9 Immunogens.

Glycine linker containing haptens for use in the vaccines were conjugated to KLH using carbodiimide conjugation chemistry as described previously^{5d, 6b}. For use as coating antigen in ELISA assays, haptens were conjugated to bovine serum albumin (BSA) or chicken albumin (OVA). Molar hapten:protein conjugation ratios (moles of hapten conjugated per mole of protein) for the BSA and OVA conjugates were quantitated by mass spectrometry (Reflex III, Bruker), as described previously^{6b}.

Conjugation of 8 and 10 Immunogens.

Sulfhydryl-containing haptens 3 and 5 were conjugated to maleimide activated BSA (mBSA) and KLH (mKLH) as described before with minor modifications^{11b}. BSA and KLH (Thermo Scientific, Rockford, Ill.) were activated with sulfosuccinimidyl-4-(N-maleimidomethyl)cyclohexane-1-carboxylate (maleimide moles 200×: BSA moles) (sulfo-SMCC, Thermo Scientific) in de-gassed 50 mM phosphate buffered saline (PBS) containing 1 mM ethylenediaminetetraacetic acid (EDTA, Sigma) for 1 hr at room temperature under nitrogen (N₂) purge to avoid oxidation. Maleimide-activated proteins were purified by dialysis in de-gassed 0.05 mM PBS under N₂ for 4 hr and stored at +4° C. The 6OXY(SH) (3) and 8HYDROC(SH) (5) haptens were dissolved in 1 ml of de-gassed 50 mM PBS, 1 mM EDTA containing 15 mM TCEP (Sigma) and added to 2 mg of mBSA or mKLH in a final 2 ml reaction volume. For 6OXY(S)-mBSA the following hapten to protein molar ratios were used to determine the optimal reaction conditions: 1,000×; 825×; 496×; 331× and 165×. For 6OXY(S)-mKLH (8) the following hapten to protein molar ratios were used to determine the optimal reaction conditions: 33,500×; 11,200×; 5,600×; 2,800×; 00×. Reactions were stirred for 2 hr at room temperature in glass vials sealed with N₂ followed by dialysis in de-gassed 0.05 mM PBS under N₂ for 6 hr and stored at +4° C. All 6OXY(S)-mKLH (8) conjugation conditions were replicated using commercially available mKLH (Thermo Fisher) to compare the immunogenicity of in-house mKLH. Conjugation of 8HYDROC(SH) (10) to BSA and KLH was based upon optimal conditions employed for the preparation of 6OXY(S)-mKLH (8).

Vaccination.

Male Holtzman rats and BALB/c mice (Harlan Laboratories, Madison, Wis.) were housed with a 12/12 hrs standard light/dark cycle. In rats, conjugates or unconjugated KLH control were injected i.p. in a final volume of 0.4 ml in complete Freund's adjuvant for the first injection and incomplete Freund's adjuvant for 2 subsequent booster injections at 3 and 6 weeks as described^{6b}. Mice were vaccinated on days 0, 14 and 28 with 25 μg of immunogen or unconjugated protein control; vaccine was administered s.c. in a final volume of 0.2 ml containing alum adjuvant as described^{5d}. Blood was obtained on day 35 for serum antibody titer measurement by facial vein sampling. All experiments were conducted 7-10 days after the 3^{rd} immunization.

Antibody.

ELISA plates were coated with 5 ng/well of BSA or OVA conjugates or unconjugated protein control in carbonate buffer at pH 9.6 and blocked with 1% gelatin. Primary antibodies were incubated with goat anti-rat IgG antibodies conjugated to horseradish peroxidase, or rabbit anti-mouse IgG antibodies to measure immunized rat and mouse sera, as described previously^{5d}.

Protein Binding of Oxycodone and Hydrocodone in Serum.

Serum protein binding of oxycodone and hydrocodone were measured as described previously^{6b}. Protein binding of oxycodone was measured in 5 rats randomly chosen in each vaccination group receiving oxycodone s.c.

Oxycodone and Hydrocodone Assay.

Serum and brain drug concentrations were measured by gas chromatography coupled to mass spectrometry as previously described^{6b}. The reported drug concentrations represent the total drug (protein or antibody-bound as well as free) in each sample.

Statistical Analysis.

Group differences were analyzed by one-way analysis of variance followed by Bonferroni post hoc test using Prism 5.0 (Graph Pad, La Jolla Ca).

### Experiment I

### Effects of Vaccination on Oxycodone Distribution after i.v. Dosing in Rats

The effects of immunization with 100 μg of the 6OXY(Gly)₄-KLH (6), 6OXY(S)-mKLH (8), 8HYDROC(Gly)₄-KLH (9), 8HYDROC(S)-mKLH (10) or unconjugated KLH control, on oxycodone distribution were first measured in rats (n=5 per group) receiving 0.1 mg/kg oxycodone administered i.v. One week after the final vaccine dose rats were anesthetized with ketamine/xylazine and an indwelling catheter was placed in their right external jugular vein. Blood was withdrawn for ELISA assays, and oxycodone administered as a 10 sec infusion. Rats were decapitated 5 minutes later and trunk blood and brain collected. The oxycodone dose was chosen to compare effects with previous reports from our laboratory^{5d, 6b}.

### Experiment II

### Effect of Vaccination on Oxycodone and Hydrocodone Distribution and Analgesia after s.c. Dosing in Rats

The effects of immunization with 100 μg of the 6OXY(Gly)₄-KLH (6), 8HYDROC(Gly)₄-KLH (9) or KLH control on oxycodone distribution and analgesia were measured at 30 min in rats (n=10 per group) receiving 2.25 mg/kg oxycodone administered s.c. This dose was previously shown to elicit a near maximal analgesic effect that was blocked by vaccination with 6OXY(Gly)₄-KLH (6)^{6b}. Hot plate analgesia tests were performed at 54° C. with 60 sec cut-offs to prevent thermal damage; hind paw licks and jumps were the predetermined behavioral endpoints to calculate % maximal possible effect (MPE)^{6b}.

In rats immunized with either 25 μg of the 6OXY(Gly)₄-KLH (6), 6HYDROC(Gly)₄-KLH (7) or KLH control (n=7-12 per group), hydrocodone analgesia was assessed after administration of 6.75 mg/kg hydrocodone s.c.; this dose was chosen through pilot studies in naïve rats to elicit a near-maximal response. In previous studies, serum antibody titers elicited from immunogen doses of 25 and 100 μg of the 6OXY(Gly)₄-KLH (6) vaccine did not differ in rats^{6b}. After completion of the hot plate behavioral endpoint, trunk blood and brain were collected to measure hydrocodone concentration.

As a specificity control for the hotplate test, fentanyl analgesia was tested using a dose of 0.3 mg/kg s.c., in a separate cohort of rats vaccinated with 6OXY(Gly)₄-KLH (6) or unconjugated KLH.

### Experiment III

### Evaluation of Vaccination on Oxycodone Analgesia after s.c. Dosing in Mice

The effects of immunization with 25 μg of either the 6OXY(Gly)₄-KLH (6), 6OXY(S)-mKLH (8), 8HYDROC(Gly)₄-KLH (9) or KLH control on oxycodone distribution and its effect on hot plate analgesia were measured in BALB/c mice (n=5-8 per group) receiving 2.25 mg/kg oxycodone administered s.c., the same dose as used for rats. Analgesia tests in mice were performed as with rats at 30 min. Hind paw lifts, hind paw licks and jumps were the predetermined behavioral endpoints to calculate the % MPE. The effect of immunization on oxycodone analgesia with the 6 vaccine was compared to several 8 conjugates to determine the most efficient sulfhydryl-maleimide conjugation conditions in BALB/c mice (n=5-7 per group). Mice received 2.25 mg/kg oxycodone s.c.

### Example 3 REFERENCES

1. (a) 2008 Department of Defense Survey of Health Related Behaviors Among Active Duty Military Personnel; 2009; (b) National Survey on Drug Use and Health: National Findings; 2011; (c) Compton, W. M.; Volkow, N. D., Abuse of prescription drugs and the risk of addiction. Drug Alcohol Depend 2006, 83 Suppl 1, S4-7; (d) Compton, W. M.; Volkow, N. D., Major increases in opioid analgesic abuse in the United States: concerns and strategies. Drug Alcohol Depend 2006, 81 (2), 103-7; (e) Maxwell, J. C., The prescription drug epidemic in the United States: a perfect storm. Drug Alcohol Rev 2011, 30 (3), 264-70.
2. (a) Oxymorphone Abuse: A growing Threat Nationwide; EWS Report 000011; 2011; (b) Epidemic: responding to America's prescription drug abuse crisis; 2011.
3. (a) Butler, S. F.; Black, R. A.; Cassidy, T. A.; Dailey, T. M.; Budman, S. H., Abuse risks and routes of administration of different prescription opioid compounds and formulations. Harm Reduct J 2011, 8, 29; (b) Dodrill, C. L.; Helmer, D. A.; Kosten, T. R., Prescription pain medication dependence. Am J Psychiatry 2011, 168 (5), 466-71; (c) Lofwall, M. R.; Moody, D. E.; Fang, W. B.; Nuzzo, P. A.; Walsh, S. L., Pharmacokinetics of Intranasal Crushed OxyContin and Intravenous Oxycodone in Nondependent Prescription Opioid Abusers. J Clin Pharmacol 2011; (d) Stoops, W. W.; Hatton, K. W.; Lofwall, M. R.; Nuzzo, P. A.; Walsh, S. L., Intravenous oxycodone, hydrocodone, and morphine in recreational opioid users: abuse potential and relative potencies. Psychopharmacology (Berl) 2010, 212 (2), 193-203; (e) Walsh, S. L.; Nuzzo, P. A.; Lofwall, M. R.; Holtman, J. R., Jr., The relative abuse liability of oral oxycodone, hydrocodone and hydromorphone assessed in prescription opioid abusers. Drug Alcohol Depend 2008, 98 (3), 191-202; (f) Wu, L. T.; Woody, G. E.; Yang, C.; Blazer, D. G., How do prescription opioid users differ from users of heroin or other drugs in psychopathology: results from the National Epidemiologic Survey on Alcohol and Related Conditions. J Addict Med 2011, 5 (1), 28-35; (g) Wu, L. T.; Woody, G. E.; Yang, C.; Mannelli, P.; Blazer, D. G., Differences in onset and abuse/dependence episodes between prescription opioids and heroin: results from the National Epidemiologic Survey on Alcohol and Related Conditions. Subst Abuse Rehabil 2011, 2011 (2), 77-88.
4. (a) Haney, M.; Gunderson, E. W.; Jiang, H.; Collins, E. D.; Foltin, R. W., Cocaine-specific antibodies blunt the subjective effects of smoked cocaine in humans. Biol Psychiatry 2010, 67 (1), 59-65; (b) Hatsukami, D. K.; Jorenby, D. E.; Gonzales, D.; Rigotti, N. A.; Glover, E. D.; Oncken, C. A.; Tashkin, D. P.; Reus, V. I.; Akhavain, R. C.; Fahim, R. E.; Kessler, P. D.; Niknian, M.; Kalnik, M. W.; Rennard, S. I., Immunogenicity and smoking-cessation outcomes for a novel nicotine immunotherapeutic. Clin Pharmacol Ther 2011, 89 (3), 392-9; (c) Martell, B. A.; Orson, F. M.; Poling, J.; Mitchell, E.; Rossen, R. D.; Gardner, T.; Kosten, T. R., Cocaine vaccine for the treatment of cocaine dependence in methadone-maintained patients: a randomized, double-blind, placebo-controlled efficacy trial. Arch Gen Psychiatry 2009, 66 (10), 1116-23.
5. (a) Anton, B.; Leff, P., A novel bivalent morphine/heroin vaccine that prevents relapse to heroin addiction in rodents. Vaccine 2006, 24 (16), 3232-40; (b) Bonese, K. F.; Wainer, B. H.; Fitch, F. W.; Rothberg, R. M.; Schuster, C. R., Changes in heroin self-administration by a rhesus monkey after morphine immunisation. Nature 1974, 252 (5485), 708-10; (c) Li, Q. Q.; Luo, Y. X.; Sun, C. Y.; Xue, Y. X.; Zhu, W. L.; Shi, H. S.; Zhai, H. F.; Shi, J.; Lu, L., A morphine/heroin vaccine with new hapten design attenuates behavioral effects in rats. J Neurochem 2011, 119 (6), 1271-81; (d) Pravetoni, M.; Raleigh, M. D.; Le Naour, M.; Tucker, A. M.; Harmon, T. M.; Jones, J. M.; Birnbaum, A. K.; Portoghese, P. S.; Pentel, P. R., Co-administration of morphine and oxycodone vaccines reduces the distribution of 6-monoacetylmorphine and oxycodone to brain in rats. Vaccine 2012, 30 (31), 4617-24; (e) Stowe, G. N.; Vendruscolo, L. F.; Edwards, S.; Schlosburg, J. E.; Misra, K. K.; Schulteis, G.; Mayorov, A. V.; Zakhari, J. S.; Koob, G. F.; Janda, K. D., A vaccine strategy that induces protective immunity against heroin. J Med Chem 2011, 54 (14), 5195-204; (f) Wainer, B. H.; Fitch, F. W.; Rothberg, R. M.; Fried, J., Morphine-3-succinyl-bovine serum albumin: an immunogenic hapten-protein conjugate. Science 1972, 176 (39), 1143-5.
6. (a) Findlay, J. W.; Butz, R. F.; Jones, E. C., Relationships between immunogen structure and antisera specificity in the narcotic alkaloid series. Clin Chem 1981, 27 (9), 1524-35; (b) Pravetoni, M.; Le Naour, M.; Harmon, T.; Tucker, A.; Portoghese, P. S.; Pentel, P. R., An oxycodone conjugate vaccine elicits oxycodone-specific antibodies that reduce oxycodone distribution to brain and hot-plate analgesia. J Pharmacol Exp Ther 2012.
7. Lewenstein, M. J., Morphine and codeinone derivatives. 1964; Vol. GB945909 19640108.
8. Glaser, M.; Karlsen, H.; Solbakken, M.; Arukwe, J.; Brady, F.; Luthra, S. K.; Cuthbertson, A., 18F-fluorothiols: a new approach to label peptides chemoselectively as potential tracers for positron emission tomography. Bioconjug Chem 2004, 15 (6), 1447-53.
9. Sondergaard, K.; Kristensen, J. L.; Palner, M.; Gillings, N.; Knudsen, G. M.; Roth, B. L.; Begtrup, M., Synthesis and binding studies of 2-arylapomorphines. Org Biomol Chem 2005, 3 (22), 4077-81.
10. (a) Kotick, M. P.; Leland, D. L.; Polazzi, J. O.; Schut, R. N., Analgesic narcotic antagonists. 1. 8 beta-Alkyl-, 8 beta-acyl-, and 8 beta-(tertiary alcohol)dihydrocodeinones and -dihydromorphinones. J Med Chem 1980, 23 (2), 166-74; (b) Sagara, T.; Okamura, M.; Shimohigashi, Y.; Ohno, M.; Kanematsu, K., Specificity affinity labeling of μ opioid receptor in rat brain by S-activated sulfydryldihydromorphine analogs. Bioorg. Med. Chem. Lett. 1995, 5 (15), 1609-1614; (c) Kolev, T.; Bakalska, R.; Seidel, R. W.; Mayer-Figge, H.; Oppel, I. M.; Spiteller, M.; Sheldricic, W. S.; Koleva, B. B., Novel codeinone derivatives via Michael addition of barbituric acids. Tetrahedron: Asymmetry 2009, 20 (3), 327-334.
11. (a) Carroll, F. I.; Blough, B. E.; Pidaparthi, R. R.; Abraham, P.; Gong, P. K.; Deng, L.; Huang, X.; Gunnell, M.; Lay, J. O., Jr.; Peterson, E. C.; Owens, S. M., Synthesis of mercapto-(+)-methamphetamine haptens and their use for obtaining improved epitope density on (+)-methamphetamine conjugate vaccines. J Med Chem 2011, 54 (14), 5221-8; (b) Pravetoni, M.; Keyler, D. E.; Pidaparthi, R. R.; Carroll, F. I.; Runyon, S. P.; Murtaugh, M. P.; Earley, C. A.; Pentel, P. R., Structurally distinct nicotine immunogens elicit antibodies with non-overlapping specificities. Biochem Pharmacol 2012, 83 (4), 543-50.
12. Janda, K. D.; Benkovic, S. J.; Lerner, R. A., Catalytic antibodies with lipase activity and R or S substrate selectivity. Science 1989, 244 (4903), 437-40.

### Example 4

### Combination of Vaccines with Medication

Drug addiction poses an increasing social and economic burden on society. Prescription opioid abuse in the United States has increased dramatically in the last decade. In one state alone, the number of prescription opioid abusers has gone up six-fold in the past five years. Treatment for prescription opioid abuse is limited to few approved medications that have sub-optimal clinical efficacy. Naltrexone (NLTX), an opioid receptor antagonist, is approved for treatment of opioid abuse and alcohol dependence. Recently, a vaccine against oxycodone (OXY) showed pre-clinical efficacy in animal models of addiction. The current study tests if NLTX blocks OXY induced analgesia in rats and if NLTX can be combined with the OXY vaccine. Experiment 1 found that NLTX blocks the acute effects of OXY in a thermal nociception test. Experiment 2 found that NLTX does not block the behavioral effects of repeated OXY administration. Experiment 3 found that the OXY-KLH vaccine may be combined with NLTX to improve its effects. Results provide proof-of-concept for combination therapy for prescription opioid abuse.

Methods

Vaccination:

Male Holtzman rats immunized once every 3 weeks for a total of 3 injections i.p., with 25 μg immunogen in Freund's adjuvant.

Antibody Characterization:

Antibody titers and concentrations measured via enzyme-linked immunosorbent assay (ELISA).

Drug Distribution Studies:

Plasma and brain collected at 33 min after behavioral assessment on the hot plate, and measured using gas chromatography coupled to mass spectrometry.

Thermal Nociception:

Rats were injected with 2.25 mg/kg OXY and placed on a hot plate for up to 60 seconds.

Results

Naltrexone Dose-Response on Acute OXY Behavioral Effects.

NLTX (0.001-0.01 mg/kg s.c.) was administered 15 min prior to 2.25 mg/kg OXY administration. Significant differences existed between saline pretreated control rats and rats receiving NLTX. For pharmacokinetic experiments NLTX dose of 0.003 mg/kg was chosen based on MPE %. *p<0.05.

Naltrexone does not Prevent Behavioral Tolerance to OXY.

In rats pretreated with NLTX, repeated OXY administration elicits decreasing MPE % similarly to rats pretreated with saline. This experiment suggests that tolerance to OXY exists and NLTX is unable to block the effects of tolerance. *p<0.05.

Vaccination Decreases Brain OXY by Increasing the Amount of OXY Sequestered in Serum.

After s.c. administration of 2.25 mg/kg, the concentration of OXY increased in serum and decreased 74% in brain of vaccinated rats versus KLH controls. *p<0.05.

Vaccination Blocks Analgesic Effects of OXY.

A) OXY-KLH does not significantly alter baseline latencies after habituation. B) Following s.c. administration of 2.25 mg/kg of OXY, vaccinated rats showed a 75% decrease in MPE % indicating decreased OXY analgesia. *p<0.05.

Naltrexone does not Interfere with Effect of Vaccination on OXY Distribution to serum.

After administration of 2.25 mg/kg OXY s.c., immunization with the OXY-KLH vaccine increased retention of OXY in serum (*p<0.05. Pre-treatment with 0.003 mg/kg NLTX does not alter the distribution of OXY in rats immunized with the OXY-KLH vaccine or the unconjugated KLH protein control. These data show that OXY-KLH is successfully able to sequester a significant amount of OXY in the serum and that NLTX does not interfere with vaccine effects. These data show that NLTX can be combined with vaccination against OXY and may be used in combination therapy with vaccination.

Conclusions

NLTX dose-dependently blocks the acute behavioral effects of OXY in a test of thermal nociception. NLTX blocks OXY-induced analgesia after repeated OXY exposure but it does not prevent tolerance to OXY behavioral effects. The OXY-KLH vaccine increases retention of OXY in serum and reduces brain OXY. Vaccination with OXY-KLH prevents the acute behavioral effects of OXY in the hot plate test of analgesia. Naltrexone can be administered in combination with vaccination, since it does not interfere with the effect of vaccination on OXY distribution in serum.

### Example 5

### Pre-Clinical Data Showing In Vivo Efficacy of Vaccination Against Fentanyl

Measuring blockage of fentanyl analgesia in vaccinated subjects is a high-throughput screening to test the vaccine's efficacy in preventing fentanyl behavioral effects. Blockage of acute opioid antinociceptive effects correlated well with vaccine efficacy in preventing drug self-administration.

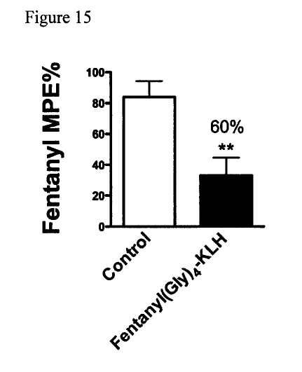

As shown in FIG. 15, in rats immunized with a fentanyl hapten conjugated to the KLH carrier protein, the antinociceptive (i.e., analgesic) effects of fentanyl (0.05 mg/kg, s.c.) were reduced by 60% compared to control rats immunized with the unconjugated KLH.

### Example 6

### Vaccination with the OXY-KLH Immunogen Prevents Oxycodone Intravenous Self-Administration in Rats

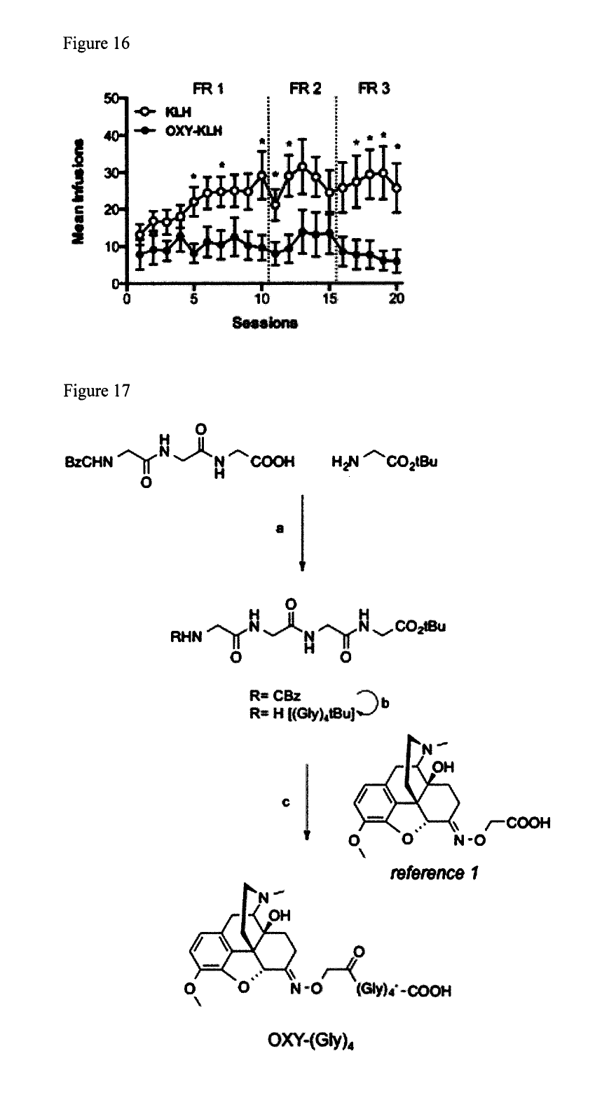

Intravenous drug self-administration is the gold standard behavioral assay to test the pre-clinical efficacy of addiction medications. In this study, the inventors tested the ability of the OXY-KLH vaccine to prevent oxycodone intravenous self-administration in rats. As shown in FIG. 16, rats immunized with the OXY-KLH vaccine showed reduced mean infusion of oxycodone compared to control rats immunized with the unconjugated KLH control carrier. During the last segment of the study, control rats immunized with KLH self-administered 29±20 infusions of oxycodone, while rats vaccinated with OXY-KLH significantly decreased (p<0.05) their drug intake down to 7±10 infusions of oxycodone (mean±standard deviation). During the entire study, control KLH rats earned a total of 501±280 infusions, while vaccinated OXY-KLH rats earned a significantly decreased total of 194±221 infusions (p<0.05).

### Example 7

### Scalable Process for OXY(Gly)₄ Synthesis

With regards to modest yields obtained with the DCC/HOBt coupling procedure (Pravetoni, M.; Le Naour, M.; Harmon, T.; Portoghese, P. S.; Pentel, P. R. An oxycodone conjugate vaccine elicits oxycodone-specific antibodies that reduce oxycodone distribution to brain and hot-plate analgesia. J. Pharmacol. Exp. Ther. 2012, 341(1), 225-232), different conditions were studied. Using HBTU with Hunig's base in DMF or DCM as solvent led to high yields and high degrees of purity. This 5 steps synthesis required only one purification by chromatography, and makes the process readily accessible for cGMP standards. FIG. 17 displays the synthesis of hapten OXY(Gly)₄ with these new conditions carried out on a 5 grams scale.

Protocols:

Linker (Gly)₄tBu: To a DMF mixture of 1 equivalent of N-protected acid component, 1.3 equivalent of coupling reagent HBTU, and 1 equivalent of the C-protected amino acid, 2 equivalent of diisopropylethylamine was added. The reaction was stirred at room temperature for 16 hours. After completion of the reaction, cold DCM was added and the precipitate was filtered and washed with DCM. The crude residue (white solid) was employed directly for the deprotection step without further purification. The deprotection reaction was carried out as previously reported to afford the desired compound as a white solid with a 93% yield overall.

OXY(Gly)₄: To a DCM mixture of 1 equivalent of carboxylic acid, 1.5 equivalent of coupling reagent HBTU, and 1.1 equivalent of the linker (Gly)₄tBu, 3 equivalents of diisopropylethylamine were added. The reaction was stirred at room temperature for 16 hours. The reaction mixture was separated from the solvent, the crude residue was taken up in ethyl acetate and washed successively three times with citric acid (5%), sodium bicarbonate (5%), and a saturated solution of sodium chloride. The organic phase was dried over Na₂SO₄, filtered and concentrated under reduce pressure. The final crude was purified by flash chromatography on silica gel using a 2% MeOH/DCM mixture to afford the desired intermediate as a white solid (89% yield).

The deprotection was carried out as previously described. After completion of the reaction, the reaction mixture was separated from the solvent and the final product was precipitated using an acetone/Et₂O mixture to afford OXY(Gly)₄ as a white solid with 87% yield.

All the analysis were consistent with the data previously reported (Pravetoni, M.; Le Naour, M.; Harmon, T.; Portoghese, P. S.; Pentel, P. R. An oxycodone conjugate vaccine elicits oxycodone-specific antibodies that reduce oxycodone distribution to brain and hot-plate analgesia. J. Pharmacol. Exp. Ther. 2012, 341(1), 225-232.)

### Example 8

### Detailed Synthesis of Haptens Directed at Fentanyl, Alfentanil, Sufentanil and Meperidine

Background and Purpose:

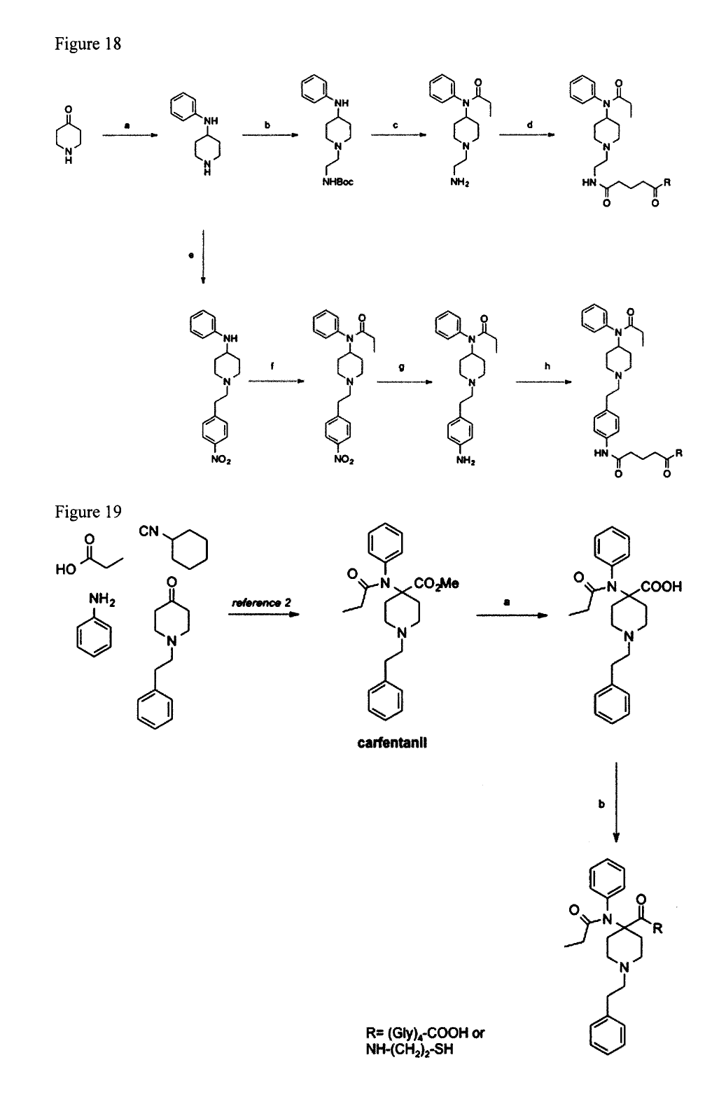

The proposed haptens are directed at fentanyl, sufentanil, alfentanil, and meperidine, which are very potent and currently marketed opiates. Three series of haptens were designed, synthesized and attached to our best linkers. FIGS. 18 and 19 display the synthetic pathways that were experimented and optimized to provide the targeted haptens.

Reactants for FIG. 18.

a. aniline, zinc, AcOH, dichloroethane, 80 C, 81%; b. 2-(N-Boc-amino)ethylbromide, K₂CO₃, CH₃CN, reflux, 87%; c. i. propionyl chloride, DMAP, DCM, r.t, 89%; ii. H+; d. i) glutaric anhydride, THF, DMAP cat., rt; e. i) Gly₄tBu or STr-cysteamine, HBTU, DIPEA, DCM, r.t.; iii) TFA/DCM (20% volume), r.t., or TFA/AcOH/DCM, r.t, 40-50 over 3 steps; e. 4-nitrophenethylbromide, K₂CO₃, CH₃CN, reflux, 93; %; f. propionyl chloride, DMAP, DCM, r.t, 87%; g. zinc, AcOH, r.t, 81%; h. i) glutaric anhydride, anh. pyridine, reflux, 20 h, 74%; ii) DCC/HOBt, Gly₄tBu or STr-cysteamine, anh. DMF, r.t, 72-83%; iii) TFA/DCM (20% volume), r.t. or TFA/AcOH/DCM, r.t.

Reactants for FIG. 19:

a. TFA, CH₃CN, 80° C., 24 h, 94%; b. i.Gly₄tBu or STr-cysteamine, HBTU, DIPEA, DCM; ii. TFA/DCM or TFA/AcOH/DCM, 81-92%, overall.

Malaquin S et al. Tetrahedron Letters 2010, 51, 2983-2985.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A multivalent composition comprising a mixture of at least two different antigenic opioid conjugates, comprising:
Hapten¹-X¹-Z¹,
Hapten²-X²-Z², and optionally
Hapten³-X³-Z³,
wherein Hapten¹ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil;
wherein Hapten² is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten³,
wherein Hapten³ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten²,
wherein X¹, X² and X³ are linking groups, and
wherein Z¹, Z² and Z³ are antigenic carrier molecules.

2. The multivalent composition of claim 1, wherein X¹ and/or X² and/or X³ is a peptide.

3. The multivalent composition of claim 2, wherein the peptide is 2-8 amino acids in length.

4. The multivalent composition of claim 2, wherein X¹ and/or X² and/or X³ is (Gly)₄.

5. The multivalent composition of claim 1, wherein Z¹ and/or Z² and/or Z³ is a protein or peptide.

6. The multivalent composition of claim 5, wherein Z¹ and/or Z² and/or Z³ is keyhole limpet hemocyanin (KLH), cholera toxin B (rCTB), recombinant diphtheria toxin CRM197, or tetanus toxoid (TT).

7. The multivalent composition of claim 1, wherein Hapten¹ is a derivative of oxycodone.

8. The multivalent composition of claim 1, wherein the multivalent composition induces antibody production in a mammal, wherein the antibodies have sufficient affinity for an opioid to alter the opioid's distribution or behavioral effects in the mammal.

9. The multivalent composition of claim 1, wherein the multivalent composition elicits antibodies that have a lower affinity for methadone, buprenorphine, naloxone, naltrexone or endogenous opioids than for their targeted opioids.

10. The multivalent composition of claim 1, further comprising an adjuvant.

11. An antigenic opioid conjugate comprising:
Hapten-X-Z
wherein the hapten is oxycodone or a derivative of heroin, 6-acetylmorphine, morphine-6-glucuronide, hydromorphone, codeine, oxymorphone, hydrocodone, fentanyl, sufentanil, meperidine, or alfentanil, or a derivative thereof;
wherein X is a linking group, and
wherein Z is an antigenic carrier molecule.

12. The antigenic opioid conjugate of claim 11, wherein X is a peptide.

13. The antigenic opioid conjugate of claim 12, wherein the peptide is 2-8 amino acids in length.

14. The antigenic opioid conjugate of claim 12, wherein X is (Gly)₄.

15. The antigenic opioid conjugate of claim 11, wherein Z is a protein or peptide.

16. The antigenic opioid conjugate of claim 11, wherein the Hapten is a derivative of oxycodone.

17. The antigenic opioid conjugate of claim 11, wherein the conjugate induces antibody production in a mammal, wherein the antibodies have sufficient affinity for opioid to alter the opioid's distribution or its behavioral effects in the mammal.

18. The antigenic opioid conjugate of claim 11, wherein the conjugate elicits antibodies that have a lower affinity for methadone than for the targeted opioid.

19. The composition of claim 11, wherein the antigenic opioid conjugate consists of
Oxycodone-X-Z
wherein X is a linking group, and
wherein Z is an antigenic carrier molecule.

20. A vaccine formulation comprising an effective amount of
(a) a multivalent composition comprising a mixture of at least two different antigenic opioid conjugates, comprising:
Hapten¹-X¹-Z¹,
Hapten²-X²-Z², and optionally
Hapten³-X³-Z³,
wherein Hapten¹ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil;
wherein Hapten² is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten³,
wherein Hapten³ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten²,
wherein X¹, X² and X³ are linking groups, and
wherein Z¹, Z² and Z³ are antigenic carrier molecules; or
(b) multivalent composition comprising a mixture of at least two different antigenic opioid conjugates, comprising:
Hapten¹-X¹-Z¹,
Hapten²-X²-Z², and optionally
Hapten³-X³-Z³,
wherein Hapten¹ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil;
wherein Hapten² is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten³,
wherein Hapten³ is a derivative of heroin, 6-acetylmorphine, morphine, morphine-6-glucuronide, hydromorphone, codeine, oxycodone, oxymorphone, hydrocodone, fentanyl, sufentanil, meperidine, or alfentanil, but is not the same compound as Hapten¹ or Hapten²,
wherein X¹, X² and X³ are linking groups, and
wherein Z¹, Z² and Z³ are antigenic carrier molecules.

21. A method of treating opioid addiction comprising administering to a mammal in need thereof the vaccine of claim 20, wherein serum level of generated antibodies is sufficient to alter the opioid's distribution or its behavioral effects in the mammal.
